(19) United States
(12) Patent Application Publication
(10) Pub. No.: US 2004/0058852 A1
(43) Pub. Date: Mar. 25, 2004

(54) Use of gap-junction blockers in neurodegenerative disease

(76) Inventors: Renato Rozental, Scarsdale, NY (US); Patric K. Stanton, New Rochelle, NY (US); David C. Spray, Pelham, NY (US); Peter L. Carlen, Toronto (CA); Christian Naus, Vancouver (CA)

Correspondence Address: Craig J. Arnold, Esq., Amster, Rothstein & Ebenstein, 90 Park Avenue, New York, NY, 10016, US

(21) Appl. No.: 10/251,640

(22) Filed: Sep. 20, 2002

(51) Int. Cl. A61K 38/00; A61K 31/40

(52) U.S. Cl. 514/2

(57) ABSTRACT

This invention relates to methods of preventing and treating hypoxic-ischemic injury and the consequences of that condition in the newborn utilizing a class of drugs known as the gap junction blockers. The invention provides a method for treating a preterm or term newborn infant with suspected or diagnosed hypoxic-ischemic injury with a gap junction blocker either before or after delivery. The present invention also provides a method for preventing seizures in term or preterm newborn infants. A method for treating and preventing the consequences of head injury is also described.

### BACKGROUND OF THE INVENTION

1. Field of the Invention

The present invention generally relates to treatments for acquired encephalopathy. More specifically, the invention relates to methods of preventing and treating hypoxic-ischemic cerebral injury and the consequences of that condition.

2. Description of the Related Art

American Academy of Pediatrics: Relation between perinatal factors and neurological outcome. In: Guidelines for Perinatal Care. 3rd ed. Elk Grove Village, 111: American Academy of Pediatrics, 221-234. (1992)

Altman, J. & Das, G. D. Autoradiographic and histological evidence of postnatal hippocampal neurogenesis in rats. J. Comp. Neurol. 124, 319-335 (1965).

Bjelke, B., Andersson, K., Ogren, S. O. & Bolme, P. Asphytic lesion: proliferation of tyrosine hydroxylase- immunoreactive nerve cell bodies in the rat substantia nigra and functional changes in dopamine neurotransmission. Brain Res. 543, 1-9 (1991).

Brake, W. G., Boksa, P. & Gratton, A. Effects of perinatal anoxia on the acute locomotor response to repeated amphetamine administration in adult rats, Psychopharmacol. (Berl) 133, 389-395 (1997)

Carlen, P. L., et al. The role of gap junctions in seizures. Brain Research Reviews 32, 235-241 (2000).

Cepeda, C., Walsh, J. P., Peacock, W., Buchwald, N. A., & Levine, M. S. Dye-coupling in human neocortical tissue resected from children with intractable epilepsy. Cerebral Cortex 3, 95-107 (1993).

Cheng, Y. et al. Caspase inhibitor affords neuroprotection with delayed administration in a rat model of neonatal hypoxic-ischemic brain injury. J. Clin. Invest. 101, 1992-1999 (1998).

Cotrina, M. L et al. Astrocytic gap junctions remain open during ischemic conditions. J. Neurosci. 18, 2520-2537 (1998).

DeRobertis and DeRobertis. Cell and Molecular Biology. Seventh Edition. 1980. Saunders College, Philadelphia.

Elshami, A. A. et al. Gap junctions play a role in the “bystander effect” of the herpes simplex virus thymidine kinase/ganciclovir system in vitro. Gene Ther. 3, 85-92 (1996).

Frade, J. M., Rodriguez-Tebar, A. & Barde, Y. A. Induction of cell death by endogenous nerve growth factor through its p75 receptor. Nature (London) 383, 166-168 (1996).

Frantseva, M. V., Kokarovtseva, L. & Perez Velazquez, J. L. Ischemia-induced brain damage depends on specific gap-junctional coupling. J. Cerebral Blood Flow and Metabol. 22, 453-462 (2002a).

Frantseva, M. V. et al. Specific gap junctions enhance the neuronal vulnerability to brain traumatic injury. J. Neurosci. 22, 644-653 (2002b).

Gage, A. T. & Stanton, P. K. (1996) Hypoxia triggers neuroprotective alterations in hippocampal gene expression via a heme-containing sensor. Brain Res. 719, 172-178 (1996).

Guyot, L. L., Diaz, F. G., O'Regan, M. H., Song, D. & Phillis, J. W. Topical insulin and accumulation of excitotoxic and other amino acids in ischemic rat cerebral cortex. Proc. Soc. Exp. Biol. Med. 224, 8-31 (2000).

Hall R T, Hall F K, Daily D K. High-dose phenobarbital therapy in term newborn infants with severe perinatal asphyxia: a randomized, prospective study with three-year follow-up. J. Pediatr. 132, 345-348 (1998).

Homma, N. et al. A particle-receptor model for the insulin-induced closure of connexin43 channels. Circ. Res. 83, 27-32 (1998).

Jellinck, P. H., Monder, C., McEwen, B. S. & Sakai, R. R. Differential inhibition of 11 betahydroxysteroid dehydrogenase by carbenoxolone in rat brain regions and peripheral tissues. J. Steroid Biochem. Mol. Biol. 46, 209-213 (1993).

Jacinto, S. J., Gieron-Korthals, M. & Ferreira, J. A. Predicting outcome in hypoxic-ischemic brain injury. Pediatr. Clin. North Am. 48(3), 647-660 (2001).

Legido, A. et al. Perinatal Hypoxic Ischemic Encephalopathy: Current and Future Treatments. International Pediatrics. 15, 143-157 (2000).

Lin, J. H. et al. Gap junction-mediated propagation and amplification of cell injury. Nat. Neurosci. 1, 494-500 (1998).

Lipton, P. Ischemic cell death in brain neurons. Physiol. Rev. 79, 1431-1568 (1999).

Lucey, J F. Neuroprotection and perinatal brain care: the field of the future, currently going nowhere. Pediatrics 100, 1030-1031 (1997).

Nelson K B, Grether J K. Can magnesium sulphate reduce the risk of cerebral palsy in very low birth weight infants? Pediatrics 95, 263-269 (1995).

Nakajima, W. et al. Apoptosis has a prolonged role in the neurodegeneration after hypoxic ischemia in the newborn rat. J. Neurosci. 20, 7994-8004 (2000).

Pitts, J. D. Cancer gene therapy: a bystander effect using the gap junction pathway. Mol. Carc. 11, 127-130 (1994).

Personius, K. E & Balice-Gordon, R. J. Loss of correlated motor neuron activity during synaptic competition at developing neuromuscular system. Neuron 31, 395-408 (2001).

Reyes, M., Reyes, A., Opitz, T., Kapin, M. A. & Stanton, P. K. Eliprodil, a non-competitive, NR2B-selective NMDA antagonist, protects pyramidal neurons in hippocampal slices from hypoxic/ischemic damage. Brain Res. 782, 212-218 (1998).

Rawanduzy, A., Hansen, A., Hansen, T. W. & Nedergaard, M. Effective reduction of infarct volume by gap junction blockade in a rodent model of stroke. J. Neurosurg. 87, 916-920 (1997).

Rami, A., Volkmann, T. & Winckler, J. Effective reduction of neuronal death by inhibiting gap junctional intercellular communication in a rodent model of global transient cerebral ischemia. Exp. Neurol. 170, 297-304 (2001).

Rozental, R. et al. Temporal expression of neuronal connexins during hippocampal ontogeny. Brain Res. Reviews 32(1), 57-71 (2000).

Rozental, R., Srinivas, M. & Spray, D. C. How to close a gap junction channel: Efficacies and potencies of uncoupling agents. Methods Mol. Biol. 154, 447-476 (2001a).

Rozental, R. et al. Gap junction-mediated bidirectional signaling between human fetal hippocampal neurons and astrocytes. Dev. Neurosci. 23, 420-431 (2001b).

Rozental R., et al. Gap junction blockade attenuates ischemic injury in hippocampal organotypic slice cultures. Society for Neuroscience Abstract 27 (2001c).

Rice, J. E., Vannucci, R. C. & Brierley, J. B. The influence of immaturity on hypoxic-ischemic brain damage in the rat. Ann. Neurol. 9, 131-141 (1981).

Scheepens, A. et al. Alterations in the neural growth hormone axis following hypoxicischemic brain injury. Brain Res. Mol. Brain Res. 68, 88-100 (1999).

Shinoura, N. et al. Protein and messenger RNA expression of connexin 43 in astrocytomas: implications in brain tumor gene therapy. J. Neurosurg. 84, 839-845 (1996).

Sidhu, R. S., Tuor, U. I., & Del Bigio, M. R. Nuclear condensation and fragmentation following cerebral hypoxia-ischemia occurs more frequently in immature than older rats. Neuroscience Lett. 223, 129-132 (1997).

Siman, R., Bozyczko-Coyne, D., Meyer, S. L. & Bhat, R. V. Immunolocalization of caspase proteolysis in situ: evidence for widespread caspase-mediated apoptosis of neurons and glia in the postnatal rat brain. Neuroscience 92, 1425-1442 (1999).

Stanton, P. K. & Moskal, J. R. Diphenylhydantoin protects against hypoxia-induced impairment of hippocampal synaptic transmission. Brain Res. 546, 351-354 (1991).

Stoppini, L., Buchs, P. A. & Muller, D. A simple method for organotypic cultures of nervous tissue. J. Neurosci. Methods 37, 173-182 (1991).

Schmitz, D. et al. Axo-axonal coupling: a novel mechanism for ultrafast neuronal communication. Neuron 31, 831-840 (2001).

Towfighi, J., Zec, N., Yager, J., Housmann, C. & Vannucci, R. C. Temporal evolution of neuropathologic changes in an immature rat model of cerebral hypoxia: a light microscopic study. Acta Neuropathol. (Berl) 90, 375-386 (1995).

Trescher, W. H., Lehman, R. A & Vannucci, R. C. The influence of growth retardation on perinatal hypoxic-ischemic brain damage. Early Hum. Dev. 21,165-173 (1990).

Travagli, R. A., Dunwiddie, T. V. & Williams, J. T. Opioid inhibition in locus coeruleus. J. Neurophys. 74, 518-528 (1995).

Welberg, L. A., Seckl, J. R. & Holmes, M. C. Inhibition of 11 beta-hydroxysteroid dehydrogenase, the foeto-placental barrier to maternal glucocorticoid, permanently programs amygdala GR and mRNA expression and anxiety-like behavior in the offspring. Eur. J. Neurosci. 12, 1047-1054 (2000).

Wade, M. H., Trosko, J. E. & Schindler, M. A fluorescence photobleaching assay of gap junction-mediated communication between human cells. Science 232, 525-528 (1986). [ 0049] Wang, J.M. et al. Reduction of ischemic brain injury by topical application of insulin-like growth factor-I after transient middle cerebral artery occlusion in rats. Brain Res. 859, 381-385 (2000).

Willecke, K. et al. Structural and functional diversity of connexin genes in the mouse and human genome. Biol. Chem. 383, 725-737 (2002).

Vornov, J. J., Tasker, R. C. & Coyle, J. T. Delayed protection by M801 and tetrodotoxin in a rat organotypic hippocampal culture model of ischemia. Stroke 25, 457-464 (1994).

U.S. Pat. No. 6,251,931.

Hypoxic-Ischemic Encephalopathy

Hypoxic-ischemic encephalopathy (HIE), an acquired encephalopathy caused by asphyxia, is a serious condition causing significant mortality and long-term morbidity in the newborn. “Hypoxic-ischemic encephalopathy” (HIE), the term recommended by the American Academy of Pediatrics and the American College of Obstetricians and Gynecologists, describes the clinical and pathologic condition affecting newborns without implying the timing of hypoxic insult. The incidence of perinatal asphyxia is about 1.0-1.5% in most centers.

HIE is the most common cause of neurologic disease in the newborn. The immediate effects involve a number of organ systems and include altered mental status, coma, seizures, renal dysfunction and respiratory difficulties. The course of HIE is variable. Long-term neurological morbidity and outcome is difficult to predict and is associated with a history of maternal problems during labor and delivery, a low Apgar score, a severely depressed level of consciousness, respiratory disturbances and seizures. There is a wide range of clinical presentations and it is possible for a full-term infant with significant cerebral injury to be asymptomatic in the early neonatal period.

The cause of HIE is asphyxia, defined by the American Academy of Pediatrics and the American College of Obstetricians and Gynecologists by a combination of clinical and laboratory data that includes (i) profound metabolic or mixed acidemia (pH<7.00) measured in an umbilical artery blood sample, (ii) persistence of an Apgar score of 0-3 for longer than 5 minutes, (iii) neonatal neurological sequellae (e.g. seizures, coma, hypotonia) and (iv) multiple organ involvement (e.g. kidney, heart, liver).

There is no specific diagnostic test for HIE and both clinical and laboratory data are necessary to establish a diagnosis and assess the severity of the condition. Laboratory studies include umbilical artery pH measurement, serum electrolytes, renal function studies, blood cultures and blood and cerebro-spinal fluid lactate levels. Other imaging studies, important to assess cerebral edema and exclude underlying brain disorders such as hemorrhage and congenital malformations, include head ultrasonography, cranial CT scan, MRI and Magnetic Resonance Spectroscopy. Particularly early in the course of the disease, imaging studies may not show abnormal findings and thus a normal study at an early stage of the disease cannot be used to rule out HIE. Other tests of cerebral function include electroencephalography and evoked potential testing.

HIE is defined clinically as mild, moderate or severe. Mild HIE is characterized by a slight increase in muscle tone, brisk deep tendon reflexes and transient behavioral abnormalities such as poor feeding, excessive crying and irritability. Moderately severe HIE is characterized by lethargy, significant hypotonia, diminished deep tendon reflexes, sluggish or absent neonatal reflexes, occasional bouts of apnea and seizures. The features of severe HIE include stupor or coma, respiratory compromise usually requiring ventilatory support, generalized hypotonia, absent neonatal reflexes, autonomic signs such as irregularities of heart rate and blood pressure, brainstem features such as dilated or fixed pupils poorly reactive to light and early onset seizures.

Hypoxic-ischemic injury to the prenatal and perinatal brain is a major cause of long-term morbidity and mortality in the newborn. The neurological consequences of hypoxic-ischemic injury include mental retardation, behavioral disorders, developmental delay, cerebral palsy and intractable epilepsy, conditions for which there is currently no effective therapy. Only 10% of infants who survive severe HIE are considered normal with 80% developing severe complications. In moderately severe HIE, 30-50% of survivors have serious long-term complications. Long-term functional impairments may be noted despite the absence of obvious neurological deficits. For example, among the survivors of moderately severe HIE who reach school age, 15-20% have significant learning disabilities. In severe HIE, the mortality rate is as high as 50%, with half the deaths occurring in the first month of life.

Several conditions may mimic HIE and must be excluded in the clinical setting. Newborns with severe central nervous system developmental malformations may show the perinatal depression and low Apgar scores that characterize HIE. Administration of certain drugs to the mother during labor can result in central nervous system depression in the newborn which in turn produces the clinical features of HIE such as decreased arousal, hypotonia, respiratory failure and seizures. Most of the symptoms of HIE can be noted in severe newborn infections such as bacterial sepsis, encephalitis, enteroviral infection or meningitis. A drug withdrawal syndrome may produce hyperactivity and jitteriness suggesting mild HIE. Various myopathies may produce hypotonia and a depressed appearing infant e.g. spinal muscular atrophy, myotonic dystrophy. The significant consequences of HIE mean that the clinician cannot hold off on treatment and must aggressively institute the therapeutic modalities directed towards HIE even while a definitive etiological diagnosis is pending.

HIE is considered an evolving process of injury occurring in two phases. In the first phase, the primary triggering events for HIE are brain hypoxia (a drop in the partial pressure of oxygen), ischemia (reduced cerebral blood flow) and hypercapnia (an increase in the partial pressure of carbon dioxide). Following the onset of hypoxia early compensatory adjustments are activated. There is an initial increase in Cerebral Blood Flow (CBF) in an attempt to increase oxygen delivery. If the asphyxia continues, the early compensatory mechanisms begin to fail and CBF autoregulation is lost leading to “pressure passive” brain perfusion. As systemic blood pressure falls, CBF reaches critically low levels and brain hypoxia occurs. This in turn produces neuronal damage. The extent of neurological damage is determined by the nature, severity and duration of the primary injury.

The second or delayed injury phase is characterized by reperfusion injury and apoptosis (a process of programmed cell death) which may continue for days to weeks.

Neuronal damage in HIE is caused by a combination of cerebral blood flow dysregulation, metabolic factors such as increased anerobic metabolism, decreased ATP, hypoglycemia and hyperlactacidemia. At a cellular level pathological processes include an increase in the excitatory amino acids glutamate and aspartate, intracellular accumulation of calcium, dysfunction of calcium-binding proteins, activation of nitric oxide synthesis and production of free radicals.

The excitatory amino acids glutamate and aspartate cause neuronal death through activation of receptor subtypes such as kainate, N-methyl-D-aspartate (NMDA), and amino-3-hydroxy-5-methyl-4 isoxazole propionate (AMPA). The high intracellular concentration of calcium, the consequence of activation of receptors with associated ion channels such as NMDA, leads to cell death. Excitatory amino acids may also contribute to the second phase of injury by disrupting factors that normally control apoptosis thereby increasing the rate of programmed cell death.

Following an episode of asphyxia, the neurons most affected die within hours while those on the periphery of the affected area may remain viable for days before succumbing to the injury. This indicates that an intervention that prevents the spread of neuronal death would reduce the severity of HIE in a newborn infant.

The eventual magnitude of neuronal damage is further influenced by the presence of underlying factors such as intrauterine growth retardation or preexisting brain pathology. Seizures, which are frequently associated with the clinical syndrome of HIE, produce further impairment of energy metabolism and neuronal integrity, thereby exacerbating the asphyxia-induced brain injury. The development of neuroprotective therapies whose goal is to prevent neuronal damage is based on counteracting these pathophysiological mechanisms.

Areas of the brain affected in HIE vary considerably and are related to factors such as the cause of the HIE, brain maturity at the time of the insult and the gestational age of the infant. The patterns of injury include parasagittal cerebral necrosis, status marmoratus, focal and multifocal ischemic brain necrosis and periventricular leukomalacia.

HIE is the most frequent cause of seizures in the newborn. The seizures usually occur from 12-24 hours after birth. The seizures can be focal, multifocal or generalized with the clinical features being motor, tonic, myoclonic or apnea. Because of the difficulty in recognizing and diagnosing seizures in the newborn, the exact incidence is uncertain, but clinically evident seizures are estimated to occur in at least 0.5% of all newborns (Holden et al., 1982). Seizures may cause or exacerbate brain injury in newborns following hypoxic-ischemic insults as a consequence of the impairment of energy metabolism and neuronal integrity. Neonatal seizures occurring in the context of HIE are correlated with poor neurodevelopmental outcome.

Gap Junction Physiology

Gap junctions, specializations of cell surfaces, mediate electrical synchrony and metabolic cooperation between apposed cells by providing conduits for the exchange of ions and small molecules (≦1 kDa) by diffusion directly from one cell to another. In a thin section, the gap junction appears as a plaque-like contact in which the plasma membranes of adjacent cells are in close approximation, separated by a space of only 2 to 4 nm. Gap junctions appear to be resistant to mechanical disruption, proteolysis and removal of calcium. Cell-cell coupling is widespread in both the immature and adult brain.

Gap junction channels are built up by docking of two hemi-channels, one from each coupled cell. The protein structure of the gap junction hemi-channel spans both the bilayer of each of the two connected cell membranes and the gap between them thereby creating a connection for intercellular communication by forming conduits between their cytoplasmic compartments (DeRobertis and DeRobertis, 1980). Each hemi-channel is composed of six protein subunits, called connexins (Cx). So far, twenty connexin subtypes have been identified in rodents, at least nine of which are expressed in the brain (Cx26, Cx30, Cx32, Cx33, Cx36, Cx37, Cx40, Cx43, Cx45 and Cx47). For most of them, orthologs in the human genome have been found.

Two connexins are likely to be the major players in neurons and astrocytes: Cx36 is the specific connexin subtype expressed in CNS neurons (Rozental et al., 2000), while Cx43 accounts for approximately 95% of all functional gap junction channels expressed in cortical astrocytes.

Cx36 expression in the central nervous system (CNS) appears to be restricted to neurons. In situ hybridization and immunocytochemical studies show high expression of Cx36 in the neurons of the inferior olive, olfactory bulb, retina, anterior pituitary, pineal gland, cerebellum, striatum, cerebral cortex (layers II-VI) and hippocampus. In situ hybridization and immunocytochemical studies show high expression in CNS regions, including the hippocampus. In the hippocampus, Cx36 is expressed in the subiculum, CA1 (stratum oriens, pyramidale, radiatum), CA2, CA3 (stratum oriens, pyramidale, radiatum) and dentate gyrus (hilus and molecular layer). Although other connexin mRNAs have been reported to be expressed in CNS neurons, including Cx32, only Cx36 protein has been confirmed as localized to neuronal gap junctions.

Cx43 expression has been firmly established in astrocytes. It appears to be present at virtually all astrocytic gap junctions and it is widely distributed in the CNS. Recent studies support a role for interastrocytic gap junctions in cell death (Lin et al., 1998; Rami et al., 2001). It is well established that astrocytes in vivo express Cx43. It appears to be present at virtually all astrocytic gap junctions and it is widely but heterogeneously distributed in the CNS, suggesting regional variation in astrocytic gap junction density. Such heterogeneity is supported by correlations between Cx43 protein expression and counts of Cx43-positive puncta among different brain regions or different fields of the same structure, including the hippocampus. Although it is known that channels formed by Cx43 and Cx36 exhibit quite different functional properties, the implications of their cellular and regional differences for CNS function and disease have not yet been investigated. Numerous roles have been postulated for gap junctions in the brain. Intercellular communication mediated by gap junctions mediates direct exchange of ions and signaling molecules between cells thereby synchronizing electrical activity between neurons and metabolic activity within astrocytes, neurons and from one cell type to another. Because of the large size of the gap junction channel pore, the scope of permeant molecules extends beyond the common intracellular inorganic ions to include amino acids, nucleotides, small peptides and sugars ranging in molecular weight up to approximately 1 kD. Preliminary studies indicate that apoptotic and necrotic signals may spread from injured cells to their neighbors through gap junction channels (i.e., “bystander” cell killing), amplifying the extent of brain ischemic injury over time. It is also thought that cytotoxic molecules produced in dying cells can kill surrounding cells by diffusion via gap junctions.

Gap junction expression in the Central Nervous System is not only region and cell specific, but also developmentally regulated (Carlen et al., 2000; Rozental, et al., 2000). Each gap junction formed by a specific connexin has different biophysical and modulatory properties such as different gating mechanisms, different permeability to ions and small molecules and different unitary conductances (Carlen et al., 2000).

Although it is known that channels formed by Cx43 and Cx36 exhibit quite different functional properties, the implications of their cellular and regional differences for CNS function and disease have not yet been investigated. Compared to Cx36, Cx43 channels are more sensitive to low pH-induced channel closure.

Gap junction channels formed by Cx43 and Cx36 exhibit strikingly different functional properties, including unitary junctional conductance, transjunctional voltage and sensitivity to intracellular pH. Compared to Cx36, Cx43 channels are more sensitive to low pH-induced channel closure. Depolarization by high extracellular K⁺ concentration, a component of ischemic insults, has been shown to increase the strength of coupling between astrocytes by activating CaM kinase pathways. In addition, ischemia results in intracellular acidification which, among other events, activates phosphatases and proteases.

Gap junctions mediate bidirectional communication between fetal neurons and astrocytes through expression of junctions composed of Cx43 in both cell types and Cx36 exclusively in neurons. As a consequence, apoptotic and necrotic signals may spread from injured cells to their neighbors though gap junction channels, amplifying the extent of brain ischemic injury over time.

Gap junctions mediate signaling between cultured neurons and between astrocytes (Rozental et al., 2000, 2001b). Neuronal-glia coupling has had a controversial history, but functional bidirectional electrical and dye coupling has recently been convincingly demonstrated (Rozental et al., 2001b). RT-PCR analysis of mRNA from hippocampal tissue and single-cell PCR revealed the expression of Cx36 and Cx43 in neurons and Cx43 in astrocytes (Rozental et al., 2001b). Neuron-to-astrocyte signaling via gap junctions is bidirectional and could account in part for the pathophysiology of ischemic-mediated brain injury.

Pyramidal neurons in the CA1 subfield are quite vulnerable to delayed neuronal death occurring 24-48 hours following ischemia. Interestingly, this time period coincides with upregulation of Cx43 mRNA expression both in vitro and in vivo in the hippocampus following HI insults. Moreover, gap junction blockade reduces the extent of injury in the field CA1 which is the basis for the hypothesized contribution of gap junctions to delayed neurotoxicity.

In studies of damage caused by stroke, gap junction channels were postulated to provide the pathway by which cytotoxic molecules spread from dying ischemic cells to nonischemic neighbors, a phenomenon termed “bystander” cell killing (Pitts, 1994). Evidence for such a role in stroke includes the neuroprotective effects of agents able to close gap junctions, including insulin (Homma et al., 1998), insulin-like growth factor-1 and growth hormone, (Scheepens et al., 1999) and reduced infarct size when the relatively nonselective gap junction blocker octanol was applied before the induction of stroke (Rawanduzy et al., 1997; Rami et al., 2001). However, there is no demonstration in the literature that gap junctions are responsible for increasing neuronal death following HIE.

Thus, gap junctions participate in propagation and amplification of neuronal damage and death following hypoxic-ischemic insults to the developing brain. Cells injured by hypoxic-ischemic insults are linked to surrounding, less affected cells, and that this “bystander-effect” expands the volume of injury over time. During brain maturation, human neurons remain extensively coupled by gap junctions; the incidence of neuronal coupling at the second-trimester of pregnancy is similar to that observed during early postnatal development (Rozental et al., 2001b; Cepeda et al., 1993).

Consistent with the belief that gap junctions play important roles in HI-induced delayed neuronal death, it has been shown that introducing connexins into various cell lines enhances bystander cell death (Elshami, A. A. et al., 1996; Shinoura, N. et al., 1996). Moreover, both pharmacological and antisense blockade of gap junctions in hippocampal organotypic slice cultures decrease neuronal vulnerability of hippocampal slice cultures to traumatic injury and to HI (Frantseva et al., 2002a; Frantseva et al., 2002b) and gap junction blockade reduces infarct size following arterial occlusion (Rawanduzy et al., 1997).

For gap junction channels, no high-affinity and highly selective inhibitor has been identified as in the case of other ion channels whose activation, inactivation and desensitization has been studied by means of highly specific toxins and the development of pharmacological agents. The agents currently available that reduce intercellular coupling via gap junctions were discovered as side effects of drugs that had been shown to affect other ion channels and of these only a very few are in general use (Rozental et al., 2001).

A number of pharmacological agents have been reported to be neuroprotective following stroke, including insulin (Homma et al., 1998; Guyot et al., 2000), insulin-like growth factor-1 (IGF-1) ( Wang et al., 2000) and growth hormone (GH)(Scheepens et al., 1999). Interestingly, one property that these share is an ability to induce closure of gap junction channels, including those formed in the cardiovascular system.

During ischemia in the CNS, astrocytic gap junctions are known to remain open (Cotrina et al., 1998) and the gap junction blocker octanol has been reported to reduce the size of infarcts after stroke (Rawanduzy et al., 1997).

Treatment of Hypoxic-Ischemic Encephalopathy (HIE)

Effective therapeutic strategies for HIE are lacking. Without a definitive treatment, supportive care remains the cornerstone of management. Supportive care is directed towards the goal of maintaining ventilation, cerebrovascular perfusion, appropriate metabolic status and adequate blood glucose levels. Blood pressure, blood gases and acid-base status must be stabilized in their physiological range. Continuous monitoring of fluid, electrolyte and nutritional status is important. Aggressive treatment of complications such as seizures and cerebral edema is required, however a successful outcome is difficult to achieve.

Attempts to treat HIE have included various drug therapies which have been used predominantly in non-human clinical trials but with limited success. For example, phenobarbital has been tried on the theory that it may be neuroprotective by reducing cerebral metabolism and oxygen consumption. While early high-dose phenobarbital produced a 27% reduction in the incidence of neonatal seizures, this effect was considered of limited clinical importance (Legido et al., 2000).

Magnesium sulfate has been widely used in obstetric practice for the suppression of preterm labor and management of pregnancy-induced hypertension. A retrospective epidemiological study suggested that premature infants whose mothers received magnesium sulfate were less likely to develop cerebral palsy. However, randomized double-blind trials showed that magnesium sulfate did not provide a protective effect against brain damage in immature fetuses and newborn infants (Legido et al., 2000).

Allopurinol, an inhibitor of the enzyme xanthine oxidase, reduced the severity of secondary edema following focal hypoxic-ischemic insult in experimental animal models but has not been successful in clinical trials in newborn infants or those who underwent heart surgery using deep hypothermic circulatory arrest (Legido et al., 2000). Likewise, calcium channel blockers, while showing theoretical promise, have been disappointing in clinical trials both in newborns and adults following strokes (Legido et al., 2000).

Non-drug interventions such as hypothermia have been suggested as neuroprotective therapy but the effectiveness remains questionable. In the clinical situation these experimental techniques are impractical. For example, not only must hypothermia cooling be started early and maintained for up to at least 48-72 hours to be protective but significant side effects have been noted such as coagulation defects, pulmonary hypertension and worsening of metabolic acidosis.

Seizures

Seizures are the result of aberrant synchrony of large neuronal aggregates. Such synchrony can be brought about by several mechanisms, including enhancement of excitatory synaptic transmission, abnormal excitatory connections, decreased activity of inhibitory synaptic transmission, increased synchrony of inhibitory neuronal networks or extracellular space alterations. Chemical synapses are undoubtedly necessary in the generation of most, if not all, types of seizures.

An expanding body of evidence suggests that gap junctions may play an important role in several types of seizures, especially in the immature brain, where gap junction distribution and activity is widespread in both neurons and glia. In animal studies, gap junction blockers inhibit epileptiform activity in hippocampal slices (Carlen et al., 2000). It has also been argued, on the basis of theoretical considerations, that in children with epilepsy very fast EEG oscillations preceding the onset of, and perhaps initiating seizures, are likely mediated by gap junctional activity although the type and cellular location of the gap junction proteins involved remains unclear.

A successful treatment protocol for HIE-induced seizures in the newborn has also been lacking. Aggressive management of seizures is important because the impairment of energy metabolism and neuronal integrity caused by ongoing seizure activity has been suggested as a potential mechanism brain injury in newborns following perinatal hypoxic-ischemia is exacerbated. In addition, early successful treatment of seizures is necessary to prevent the development of status epilepticus (continuous, uncontrolled seizures unresponsive to anticonvulsant therapy), which in turn, may significantly compromise body functions such as pulmonary ventilation and cardiac function.

While seizures are usually clinically obvious, the treating clinician has to be aware of the possibility of “subclinical” seizures which are only identifiable by EEG recording. Standard anticonvulsants utilized in this age-group include phenobarbital, lorazepam and phenytoin but other anticonvulsants such as valproic acid or lamotrigine have been tried with inconsistent results. Furthermore, the risk of drug side effects such as hepatotoxicity has limited their use.

Not only does standard anticonvulsant treatment fail to consistently control clinical seizures in the newborn but often fails to control subclinical or electrographic seizures, further suggesting the need for more effective anticonvulsant regimens.

Traumatic Brain Injury (Head Injury)

Brain injury secondary to head trauma is also a significant problem. The incidence of traumatic brain injury (TBI) in the United States has been estimated to be between 180-220 cases per 100,000 population annually which translates into approximately 600,000 new TBIs per year. Because the brain is contained in the rigid skull, any increase, even a minor one, in the volume of the brain will produce a dramatic rise in intracranial pressure. As the intracranial pressure increases, cerebral blood flow will decrease. This in turn is followed by the loss of autoregulation of the arterioles which are crucial blood vessels providing blood flow to the brain. Secondary changes occur at a cellular level with traumatic brain injury resulting in neuronal loss. Gap junction communication appears to enhance cellular vulnerability to traumatic injury (Frantseva et al., 2002).

Therefore, in summary, protection of the human brain in the light of significant hypoxic stress remains elusive and despite strenuous efforts to understand and find solutions for the devastating brain damage produced by HIE, little has been achieved. Considering the field as a whole, neuroprotection for perinatal brain damage is “currently going nowhere” (Lucey, 1997).

### SUMMARY OF THE INVENTION

Accordingly, the present invention is based upon the discovery that carbenoxolone, a gap junction blocker, when administered either before or after the onset of hypoxic-ischemic encephalopathy, reduces subsequent neuronal damage in the hippocampus, cerebellum and neocortex. On the basis of this finding, the present invention provides methods for preventing perinatal hypoxic-ischemic cerebral injury in an infant. The methods comprise administering to a woman pregnant with the infant, or to the infant following labor and delivery, an amount of gap junction blocker effective to prevent hypoxic-ischemic cerebral injury in the infant.

The present invention is also directed to methods for preventing seizures induced by an hypoxic-ischemic injury in an infant. The methods comprise administering to a woman pregnant with the infant, or to the infant following labor and delivery, an amount of gap junction blocker effective to prevent seizures following hypoxic-ischemic injury in an infant.

Additionally, the invention is directed to methods for treating seizures induced by hypoxic-ischemic cerebral injury in a infant. The methods comprise administering to a woman pregnant with the infant, or to the infant following labor and delivery, an amount of gap junction blocker effective to treat seizures following hypoxic-ischemic injury in a preterm or term infant.

In other embodiments, the present invention is directed to a kit comprising a gap junction blocker and instructions for preventing perinatal hypoxic-ischemic cerebral injury in an infant by administering to a woman pregnant with the infant during labor or to the infant following delivery an amount of gap junction blocker effective to prevent hypoxic-ischemic cerebral injury in the infant.

In additional embodiments, the present invention is directed to methods of preventing hypoxic-ischemic injury in a neuron in an infant, the methods comprise administering to a woman pregnant with the infant during labor or to the infant following delivery an amount of gap junction blocker effective to prevent hypoxic-ischemic injury to the neuron.

In further embodiments, the present invention is directed to methods for conferring neuro-protection to an individual following head trauma. The methods comprise administering to the individual an amount of gap junction blocker effective to confer neuro-protection following head-trauma in the individual.

Additional objects of the present invention will be apparent in view of the description which follows.

### BRIEF DESCRIPTION OF THE DRAWINGS

FIG. 1 summarizes experimental results which establish that in vitro organotypic rat brain slice cultures allow ready application of controlled hypoxic-ischemia (HI) insults and assessment of long-term cell viability. FIG. 1a and 1b show representative propidium iodide (PI) (1 μM) fluorescence labeling in hippocampal slice cultures at 3 (FIG. 1a) and 7 (FIG. 1b) days in culture (DIC). FIG. 1c is a graph showing PI fluorescence intensities in field CA1 Stratum Pyramidale over the first 10 days following slicing (n=11). FIG. 1d is representative Schaffer collateral-evoked excitatory postsynaptic potentials recorded at 3 and 7 days in culture (calibration bars 10 ms/3 mV). FIG. 1e is a graph showing the mean ∓S.E.M. recovery of excitatory postsynaptic potentials dV/dT (V/s; solid bars) and peak amplitude (mV; grey bars) as a function of time in culture.

FIG. 2 summarizes experimental results which establish that gap junction blockade protects CA1 pyramidal neurons in organotypic cultures from hypoxia/hypoglycemia-induced (HI) delayed neuronal death. After 6-10 days in culture, hippocampal slices were subjected to 45 minutes of severe hypoxic insult by placement in glucose-free medium in a sealed box gassed continuously with 95% N₂/5% CO₂. Twenty-four hours later, slices were exposed to 1 μM PI for 10 minutes, then photographed. FIG. 2a shows typical low level and diffuse PI fluorescence in a control, nonischemic slice culture. FIG. 2b illustrates marked PI fluorescence in CA1, CA3 and dentate gyrus (DG) 24 hr post-HI in a representative hippocampal slice culture. FIG. 2c shows reduced PI fluorescence 24 hr post-HI in a hippocampal slice culture treated with 75 μM carbenoxolone 60 min after the end of the HI episode. FIG. 2d is a graph showing mean ±S.E.M PI fluorescence in CA1 stratum pyramidale of cultures treated with 75 μM carbenoxolone 30 min before (−30′), during (0′), or 60 min after (+60′) HI, as % of HI treatment alone (n=11-13 slice cultures). FIG. 2e is a graph of the time course of mean ±S.E.M. PI fluorescence (•) and nucleosomal DNA (□) in slice cultures (n=6 per time point) during the first 24 hr post-slicing. FIG. 2f shows HI-induced activation of caspase-3 in hippocampal slice cultures demonstrating Western blots of active p17 (17 kDa) subunit of caspase-3 from HI-treated slice cultures 24 hr post-HI (ISC) and normoxic control cultures (Control). FIG. 2g is a trace of Schaffer collateral-evoked excitatory postsynaptic potentials recorded in CA1 stratum radiatum 24 hr post-HI in untreated (Ischemia) and carbenoxolone - treated (75 μM applied immediately post-HI; ISC+Carb) slice cultures (calibration bars 10 ms/3 mV). FIG. 2h shows that fluorescence recovery after photobleaching (FRAP) in CA1 pyramidal neurons in slice cultures is increased 24 hr after HI. Slices were loaded with carboxyfluorescein diacetate (30 μM) for 45 min, then washed in drug-free medium before photobleaching. Comparison of representative fields in stratum pyramidale in Control, HI alone (ISC), and HI followed by 75 μM carbenoxolone (ISC+Carb) showing slice cultures before (top row), 2 min (middle row) and 9 min (bottom row) after photobleaching.

FIG. 3 shows photographs of rats illustrating the experimental results which demonstrate that gap junction blockade after intrauterine HI ameliorates the long-term developmental impact of HI. FIG. 3a shows that in contrast to control pups delivered by C-section which had a reddish color and displayed continuous movements (CTRL), ischemic pups were cyanotic and displayed marked hypotonia (ISC). FIG. 3b shows representative control (CTRL) and intrauterine HI-treated (ISC) littermates demonstrating that the sequellae of HI episodes included reduced growth of both size and weight. FIG. 3c shows normoxic control (CTRL) and ischemic (ISC) littermates at 21 days of age. FIG. 3d shows that carbenoxolone treatment (75 mg/kg) immediately after intrauterine HI (ISC+Carb) improves long-term development assessed by size compared to HI alone (ISC) (Calibration bar: 2 cm).

FIG. 4 shows representative micrographs from the same litter demonstrating the histological examination of long-term effects of intrauterine HI on P21 brains and the reduction of the extent of histopathological damage in the hippocampus, neocortex and cerebellum by the administration of carbenoxolone. The upper panels show photomicrographs of sections of cerebellum (FIG. 4a-f; upper panels) from control (CTRL) (FIG. 4a, b, upper panels), HI-treated (ISC) (FIG. 4c, d, upper panels), and HI plus carbenoxolonetreated (ISC+Carb; 75 mg/kg i.p.) (FIG. 4e, f, upper panels) rats at 21 days of age. HI produced marked reductions in cerebellar size (FIG. 4c, upper panels) and resulted in granule layer hypertrophy (FIG. 4d, upper panels) compared to both controls (FIG. 4a, b, upper panels) and HI plus carbenoxolone-treated (FIG. 4 e, f, upper panels) littermates. The middle panels show photomicrographs of sections of hippocampus (FIG. 4a-f; middle panels) at P21 of control (CTRL) (FIG. 4a, b, middle panels), HI (ISC) (FIG. 4c, d, middle panels) and HI plus carbenoxolone-treated (ISC+Carb) (FIG. 4 e, f, middle panels) hippocampi. Note the hypercellularity in the hilus of the hippocampus following ischemia (white arrows, FIG. 4c, d, middle panels). The lower panels show photomicrographs of neocortex at P21 of sections of neocortex from control (CTRL) (FIG. 4a, b, lower panels) and HI-treated (ISC) (FIG. 4c, d, lower panels) rats. Note the disorganization of cortical laminae (FIG. 4c, lower panels), and neurons pyknotic in appearance with damaged membranes, suggestive of apoptosis (FIG. d, lower panels, black arrows). Calibration bars: 500 μm in both upper (2.5×magnification) and lower (10× magnification) panels.

FIG. 5 shows representative western blots which show that gap junction blockade prevents activation of caspase-3 by intrauterine hypoxia, measured 24 hours after hypoxic-ischemic insult. Hippocampal slice cultures: consistent with activation of caspase-3 by intrauterine hypoxia, activated caspase-3 levels were also substantially increased in hippocampal organotypic slice cultures 24 hours post hypoxia (ISC) compared to normoxic controls (Normoxic). P1 Brain 24 hr Post-HI: western blots from three pairs of litter mates from different litters treated either with intrauterine hypoxia alone (Untr), or hypoxia followed immediately by carbenoxolone (Carb) (75 mg/kg i.p.). Carbenoxolone treatment immediately after perinatal hypoxic insult reduced the activation of the apoptosis-triggering enzyme caspase-3, providing further evidence of the protective effect of gap junction blockade.

FIG. 6 shows photographs and electroencephalographic recordings that illustrate the epileptogenic effect of intrauterine hypoxic- ischemic insults in neonatal rats 30 to 60 min following delivery and the anti-epileptic effect of gap junction blockade by carbenoxolone. FIG. 6a shows a photograph which demonstrates the seizures characterized by tonic extensions and/or “swimming-like” activity experienced by 30% of the pups subjected to ischemia (C-section+Intrauterine HI insults). FIG. 6b shows a photograph of the control group which was also delivered by caesarian-section but not subjected to an hypoxic-ischemic insult demonstrating normal posture and activity (Control C-section (no ischemia)). FIG. 6c shows a tracing of electroencephalographic activity (EEG) in a single pup with electrographic epileptiform activity before administration of carbenoxolone (EEG). FIG. 6d shows a tracing of EEG activity recorded 40 minutes after administration of carbenoxolone demonstrating the loss of electrographic epileptiform activity (+Gap junction blocker).

FIG. 7 shows photographs that demonstrate the impact of perinatal behavioral seizures on P1 rat pups following intrauterine HI insults and the anti-epileptic effect of gap junction blockade by carbenoxolone. FIG. 7a shows a photograph of a pup illustrating the absence of seizures following the administration of carbenoxolone following hypoxic-ischemic insult (A). FIG. 7b shows a photograph of the untreated rat pup following hypoxic-ischemic insult in a repetitive and prolonged behavioral seizure (B).

FIG. 8 shows a histological section showing evidence for hippocampal damage observed in P21 rats following intrauterine hypoxic-ischemic insult maintained for 15 minutes. FIG. 8 (Tunel Negative) shows a histological section of ischemic hippocampus with the omission of TdT from the TUNEL mix which acts as an “internal negative” control. FIG. 8 (DNase 1) shows a section of control hippocampus that was not subjected to hypoxic-ischemic insult used as an “internal positive” control. FIG. 8 (Ischemia) shows a histological section showing pyknotic nuclei in P21 hippocampus following global brain ischemia. FIG. 8 (Mammary gland) shows a section of the involuting mammary gland of mothers 3 days following removal of the pups, used as an additional “external positive” control and showing the round chromatin clumps typical of apoptotic cells.

### DETAILED DESCRIPTION OF THE INVENTION

The present invention is based on the discovery that gap junction blockers administered before, during or after hypoxic-ischemic insult markedly reduces neurotoxicity normally associated with HIE. This discovery makes possible the development and use of methods for preventing perinatal hypoxic-ischemic cerebral injury in infants. The methods comprise administering to a woman pregnant with an infant, or to the infant following labor and delivery an amount of gap junction blocker effective to prevent hypoxic-ischemic cerebral injury. Without being limited to any particular mechanism of action, it is believed that administration of gap junction blockers prevents the diffusion of cytotoxic molecules, produced in dying cells via gap junction channels, with the result that the infant is afforded protection from the spreading effects of an hypoxic insult.

As used herein, a gap junction blocker is a compound that inhibits passage of chemicals from one cell to another through a gap junction. Gap junction blockers are not limited to any particular mechanism of inhibiting passage of chemicals through a gap junction. Nonlimiting examples of possible mechanisms of action of gap junction blockers are: inducing closure of a gap junction, blocking a gap junction (e.g. with an antibody or aptomer that specifically binds to the gap junction proteins, connexin antibodies, polypeptides corresponding to extracellular domains of connexins, antisense oligonucleotides corresponding to specific connexin sequences), inhibition of gap junction-mediated chemical and electrical transmission, and perturbations in bulk membrane fluidity or the membrane-protein interface that effects the conformation of the membrane bound gap junction proteins. See, e.g., U.S. Pat. No. 6,251,931. Nonlimiting examples of gap junction blockers are insulin, growth hormone, antibodies or optomers that bind to gap junction proteins inhibiting passage of chemicals through the gap junction, fluothane, ethrane, heptanol and glycyrrhetinic acid derivatives. In preferred embodiments, the gap junction blockers are small molecular weight compounds (i.e. <1000 Dalton) that can cross the blood-brain-barrier. Examples include fluothane, ethrane, heptanol and glycyrrhetinic acid derivatives. In more preferred embodiments, the gap junction blocker is a glycyrrhetinic acid derivative. In the most preferred embodiments the gap junction blocker is carbenoxolone.

In a particularly useful embodiment, these methods involve administering the gap junction blocker to a pregnant woman in a high risk pregnancy. Hypoxic injury to the fetus occurs eight times more frequently in high-risk pregnancies than in normal pregnancies. Thus, administration of a gap junction blocker to a woman with a high risk pregnancy serves to protect the infant from an hypoxic insult in a situation where a complication of pregnancy is recognized before labor and delivery. Complications under this category include, but are not limited to, placenta praevia, pre-eclampsia, eclampsia, intra-uterine growth retardation, maternal infection or hemorrhage.

Once such a complication is recognized, rapid termination of the pregnancy is usually necessary either by inducing labor or caesarian section. This process further increases the likelihood of hypoxic insult to the infant. Administration of a gap junction blocker prior to or soon after initiation of the required procedure (either induction of labor or caesarian section), would serve to protect the infant. In some situations the obstetrician may decide to observe the pregnant woman but closely monitor her condition and the condition of the fetus by using standard fetal monitoring devices. If worsening of the woman's condition is recognized or the onset of fetal distress is indicated by the fetal monitors rapid termination of the pregnancy is necessary and the method of the present invention would be utilized in order to provide protection to the infant.

The gap junction blocker can be administered to the pregnant woman via any method that the obstetrician, neonatologist or pediatrician present at the delivery considers most effective and expeditious, depending on the circumstances of the delivery and the urgency with which the gap junction blocker has to be administered. For example, the gap junction blocker may be administered orally. Alternatively, particularly in situations in which the woman cannot take anything by mouth, examples of other routes of administration include, but are not limited to, intravenous, sublingual, subcutaneous, cutaneous, intranasal, intrathecal, vaginal or rectal. Oral methods of administration include, but are not limited to, preparations such as liquids, tablets, caplets, pills and suspensions. Once administered to the pregnant woman the gap junction blocker is transported to the fetus via the placenta. Methods for formulating gap junction blockers for any of these routes of administration can be established by the skilled artisan without undue experimentation.

In some situations maternal complications are not recognized until labor has started. Examples of such complications include, but are not limited to, cephalo-pelvic disproportion, placental rupture and maternal hemorrhage. In these situations, the administration of gap junction blockers to the pregnant woman (for example, by the methods outlined above) could be initiated either before the onset of labor or once labor had started.

In another useful embodiment, an amount of gap junction blocker effective to prevent hypoxic-ischemic cerebral injury can be administered to the infant either during labor or following delivery. These embodiments are particularly useful in situations where complications of the process of labor are unexpected or not recognized early enough and occur too late to reliably provide the gap junction blocker to the infant via the maternal-placental blood supply.

The method of the present invention can be utilized in either term or preterm labor and delivery. As used herein, “term” refers to the culmination of pregnancy at the end of nine months. “Preterm” refers to the culmination of pregnancy before the end of nine months. Preterm labor and delivery is especially problematic and associated with a high risk of hypoxic injury. Sometimes the duration of the pregnancy is unclear, as in situations in which little if any prenatal care has been received by the woman. In the case of poor prenatal care, the pregnancy at the time of the onset of labor may be presumed to be term yet following delivery the infant is discovered to be preterm. Thus, the present invention could be utilized to provide protection against hypoxic injury in an infant discovered after delivery to be preterm. Methods of administering gap junction blockers to the infant are as described above.

The present invention is not limited to use with any particular type of delivery. When the delivery is vaginal, the gap junction blocker can be administered to the woman prior to delivery. If it is not possible to administer the gap junction blocker to the woman before vaginal delivery is completed, the infant can receive the gap junction blocker immediately following vaginal delivery. As is well known, vaginal delivery can involve different presentations of the infant including vertex, breech or shoulder. As used herein, “presentation” refers to the position of the long axis of the fetus to that of the mother during labor.

Generally, once the head is delivered, the obstetrician suctions meconium fluid or other fluids out of the infant's mouth and trachea. In some embodiments of the invention, this process allows the administration of gap junction blocker to the infant prior to the completion of delivery, thereby ensuring early dosage. In other embodiments, the gap junction blocker can be administered following complete delivery. In some embodiments of the invention, more than one dose of the gap junction blocker is given, one to the woman prior to delivery, the other to the infant following delivery.

The present invention may also be utilized when delivery of the infant is achieved by Caesarian-section (C-section). As is well known, C-section involves an incision through the abdominal wall and the uterus for delivery of a fetus. In some embodiments of the invention, the woman is given a dose of gap junction blocker prior to the C-section when it is recognized that there is a risk of hypoxic injury to the fetus. In other embodiments, the dose of gap junction blocker is given during the C-section, for example, to the infant during the C-section once the uterus has been opened and the infant exposed, or once the infant is completely separated from the placenta and delivered. The methods are useful with term or preterm C-sections.

The methods of this invention can be utilized in situations in which the hypoxic-ischemic cerebral injury in the infant is either suspected or diagnosed. Hypoxic-ischemic injury is suspected where the pregnancy is considered high risk or a complication of labor has occurred but timely evaluation of the infant is not possible. In order not to delay administration of the gap junction blocker to the infant, it is administered either to the woman prior to delivery or to the infant during or after delivery. The circumstances surrounding labor and delivery are sufficient to enable the obstetrician to recognize the risk of hypoxic-ischemic injury.

The pathological picture of hypoxic-ischemic injury can vary. The hypoxic-ischemic injury may be a “stroke”. As used herein, “stroke” refers to the results of acute or subacute vascular lesions of the brain. The stroke may be focal, that is involve a limited area of the brain, also referred to as “focal ischemic injury”. The effect of the hypoxic-ischemic insult can also be more widespread producing a “global ischemic injury”. A second hypoxic-injury may also result, producing pathological combinations of focal and global ischemic injury. The embodiments of the present invention are effective in either focal or global cerebral injuries or in those situations in which a superimposed second hypoxic-ischemic injury occurs.

As indicated above, an infant may have sustained an hypoxic-ischemic injury without there being obvious clinical signs for the obstetrician, neonatologist or pediatrician despite concerns raised by the circumstances of the delivery. Thus in further embodiments of the invention, gap junction blockers are used where the likelihood of hypoxic-ischemic injury is considered a clinical possibility. Preferably, the diagnosis of the injury is made by clinical exam, MRI, CT scan, PET scan, X-ray, arteriography, electroencephalograph, blood gas examination, blood electrolyte examination and cerebro-spinal fluid examination. Because hypoxic-ischemic injury is a process and its effects may take several days to manifest, it may be necessary to repeat some or all of this testing in situations in which, despite early negative results, there remains a high index of suspicion. The consequences of hypoxic-ischemic injury are severe and on occasion, in one embodiment of the invention, gap junction blockers have to be administered to an infant in whom there is a high index of suspicion of an hypoxic-ischemic insult but the testing has not yet confirmed the injury.

The methods of the present invention may also be utilized when hypoxic-ischemic injury is not the result of the complications of pregnancy or labor. In some embodiments, gap junction blockers are administered to those infants in whom hypoxic-ischemic injury is suspected and there is a high index of suspicion or actual diagnosed conditions that create a risk of HIE such as anterior circulation stroke, cerebral venous thrombosis, West syndrome, lysosomal storage disease, Fabry disease, homocystinuria, MELAS, neuronal ceroid lipofuscinosis, peroxisomal disorders, hyperammonemia, hypocalcemia, methylmalonic acidemia, ornithine transcarbamylase deficiency, propionic acidemia, epileptogenic encephalopathy, intractable seizures, perinatal asphyxia, encephalitis, meningitis, birth trauma, tuberous sclerosis, congenital cerebral malformations, intracranial tumors, intracranial hemorrhage, toxoplasmosis, rubella, syphilis, cytomegallovirus, herpes, systemic metabolic disease, Lennox-Gastaut syndrome, epileptogenic encephalopathy, intractable seizures, perinatal asphyxia, encephalitis, meningitis, birth trauma, tuberous sclerosis, congenital cerebral malformations, intracranial tumors, intracranial hemorrhage, toxoplasmosis, rubella, syphilis, cytomegallovirus, herpes, systemic metabolic disease or cephalo-pelvic disproportion. In these conditions the hypoxic injury may not be recognized early on, yet due to the high suspicion further diagnostic testing as outlined above may be necessary. In some embodiments of the invention, while the results of the diagnostic testing are pending, the administration of the gap junction blocker is indicated due to the high risk of hypoxic-ischemic injury. When required, the gap junction blocker may be administered to either the woman, the infant or both by a number of routes as described above.

In some embodiments of the invention, the gap junction blocker is administered for the prevention of seizures in the newborn. Seizures are a frequent clinical manifestation of hypoxic-ischemic injury and may aggravate the underlying pathological process. Aggressive management of seizures is necessary to prevent further injury to the neonatal brain.

The methods of the present invention may be utilized for the treatment of seizures. When the physician suspects that an hypoxic-ischemic insult has or is likely to occur, seizures are a likely consequence. In some embodiments of the invention, the gap junction blocker can be administered to the mother either prior to the onset of labor (natural or induced), or if the process of delivery has already started, during delivery. The methods of the invention, can be utilized in term or preterm labor. The gap junction blocker can be administered by a number of methods as described above.

The methods of the present invention can be utilized following the recognition of the likelihood of seizures in the newborn infant or the actual occurrence of seizures in the infant. In some embodiments of the invention, the administration of the gap junction blocker would be undertaken either during delivery or following completion of either term or preterm delivery by methods described above.

Seizures occurring in the population to which the invention is directed may be obvious, manifesting clinically as focal, partial, generalized, tonic, atonic, myoclonic, astatic, bicycling, minor motor, apnoeic seizures or any combination of these clinical types. In other situations the seizures may not be clinically obvious but noted only with testing by electroencephalography (EEG). For example, an infant may be paralyzed to achieve easier ventilation, and with clinical movements suppressed, clinical or behavioral seizures would not be noticed. In other situations, following high doses of anticonvulsant medication, the infant may show very limited clinical movements yet continue to have electrographic or subclinical seizures only identifiable by EEG. As used herein, “electrographic” or “subclinical” seizures refer to seizure activity that is not clinically apparent yet remains ongoing in the brain. These “electrographic” or “subclinical” seizures can only be identified by means of EEG. On occasion, EEG is not available and the physician has to use clinical judgment in the face of a high index of suspicion of “subclinical” or electrographic seizures. Thus, the methods of this invention can be utilized when the physician has a high index of suspicion that “subclinical” seizures were occurring.

The present invention is also directed to a kit comprising a gap junction blocker and instructions for preventing or reducing the severity of perinatal hypoxic-ischemic cerebral injury in an infant. In some aspects, the instructions can describe the administration of a gap junction blocker to a pregnant woman when there is a concern about the likelihood of hypoxic-ischemic injury either before or during labor which may be either natural or induced, or term or preterm. In other aspects, the kit would further include an apparatus for administering the gap junction blocker according to the methods described above. In additional aspects, the kit would also contain an amount of the gap junction blocker sufficient to provide a range of dosing options.

In additional embodiments, the present invention is directed to preventing hypoxic-ischemic injury in a neuron in an infant. The methods comprise administering to a woman pregnant with an infant, or to the infant following labor and delivery an amount of gap junction blocker effective to prevent hypoxic-ischemic injury to a neuron. In a preferred embodiment of the invention, the gap junction blocker is carbenoxolone.

In additional embodiments, the invention is directed to methods for neuroprotection following head trauma. The methods comprise administering the gap junction blocker to an individual who has experienced a head injury. The head injury may be associated with a skull fracture and be “open” or “closed”. Traumatic head or brain injury may have either acute, chronic or a combination of acute and chronic effects. The methods involve administering a gap junction blocker either soon after clinical effects become apparent or prophylactically when a head injury is considered significant enough to produce delayed effects. The gap junction blocker is administered in the non-acute phase if clinical symptoms are delayed. Administration of the gap junction blocker is by one of the methods described above.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### In Vitro Effects of Hypoxic-Ischemia and Carbenoxolone Administration

In this Example, we show the effect of controlled hypoxic-ischemic insults and the effect of carbenoxolone administration in organotypic hippocampal brain slice cultures.

Methods

Organotypic Hippocampal Slice Cultures.

Organotypic hippocampal slice cultures were prepared from 9- to 12-day old Sprague-Dawley rat pups (Charles River, Wilmington, Del.) of both sexes (Stoppini et al., 1991). Rats were decapitated under deep ketamine anesthesia, the hippocampi rapidly dissected out and placed immediately into ice-cold dissection medium containing: 3.5 mM KCl, 0.5 mM NaH₂PO₄, 10 mM MgSO₄, 5 mM NaHCO₃, 118 mM n-methyl-D-glucosamina, 1.1 mM KH₂PO₄, 10 mM HEPES, 20 mM D-glucose, pH 7.35, gassed with 95% O₂/5% CO₂. Transverse slices of hippocampus plus entorhinal cortex were cut using a spring-loaded grid of 20 μm diameter wires spaced 400 μm apart. Slices were immediately transferred into dissection medium, separated and placed on semi-permeable membrane tissue culture inserts (Millipore Corporation, Bedford, Mass.) in a 6-well plate, each well containing 1 ml of culture medium (50% Minimum Essential Medium, 25% Hank's balanced salt solution, 25% heat-inactivated horse serum, 13 mM HEPES, 2.5 mg/ml glucose). Cultures were maintained at 35° C. in a humidified 5% CO₂ incubator and medium was changed every 2-3 days.

After 6-10 days in culture, hippocampal slice cultures were exposed to hypoxia-ischemia (HI) by transient oxygen and glucose deprivation. Slices were transferred into another six-well plate containing 1 ml of Ringermannitol glucose-free medium per well, containing 1.25 mM CaCl₂, 0.9 mM MgSO₄, 5.4 mM KCl, 0.44 mM KH₂PO₄, 137 mM NaCl, 4.2 mM NaHCO₃, 0.35 mM NaH₂PO₄, 16.7 mM HEPES, and 27.5 mM mannitol, and placed in a hypoxic box continuously gassed with 95% N₂/5% CO₂ for 45 min. They were then returned to normal medium with carbon dioxide until measurement of neuronal death. This methodology has been shown to lower pO₂ to 20-30 mm Hg within 1-2 min of perfusion in acute slices, so we expect pO₂ levels inside the hypoxic box continuously gassed with 95% N₂/5% CO₂ to be reduced as much or more.

Carbenoxolone (75 μM) was added to the culture medium 30 minutes prior to, during and 60 minutes after application of a 45 minute hypoxic-ischemic insult. Carbenoxolone (disodium salt) was purchased from Sigma (St. Louis, Mo.), propidium iodide and carboxyfluorescein diacetate from Molecular Probes (Eugene, Oreg.). Cleaved caspase-3 antibody was purchased from Cell Signaling (Beverly, Mass.).

Propidium Iodide Fluorescence.

Media containing 1 μM PI was applied for 10 min to slices and PI-labeled dead cells imaged using FITC filters. IPLab (Signal Analytics Corp.) and NIH image analysis programs were used to quantitate total fluorescence intensity for equal regions of interest within the CA1 pyramidal cell body layer (stratum pyramidale).

Slice cultures were placed in an interface recording chamber between artificial cerebrospinal fluid (ACSF; perfusion rate 3 ml/min) and a humidified atmosphere of 95% O₂/5% CO₂. ACSF composition was: 126 mM NaCl 126, 5 mM KCl, 1.25 mM NaH₂PO₄, 2 mM MgCl₂, 2 mM CaCl₂, 26 mM NaHCO₃, 10 mM D-Glucose (pH 7.4, 33-34° C.). Extracellular excitatory postsynaptic potentials (EPSPs) were recorded (2 M NaCl-filled electrodes) in stratum radiatum and elicited by bipolar Schaffer collateral stimulation (intensities 20-200 μA), recorded using an Axopatch 200A amplifier in current-clamp mode.

Fluorescence Recovery After Photobleaching (FRAP).

Fluorescence recovery in CA1 fields of hippocampal slice cultures was evaluated as in Wade et al., 1986. Hippocampal slice cultures were loaded by 45 min application of carboxyfluorescein diacetate (30 μM). CA1 fields of view were photobleached for 2 min using the 488 nm laser line of a confocal microscope (Nikon RCM 8000) and images acquired at low power at 1 min intervals thereafter. Quantitative image analysis was performed using Polygon-Star software (Nikon).

Results and Conclusions

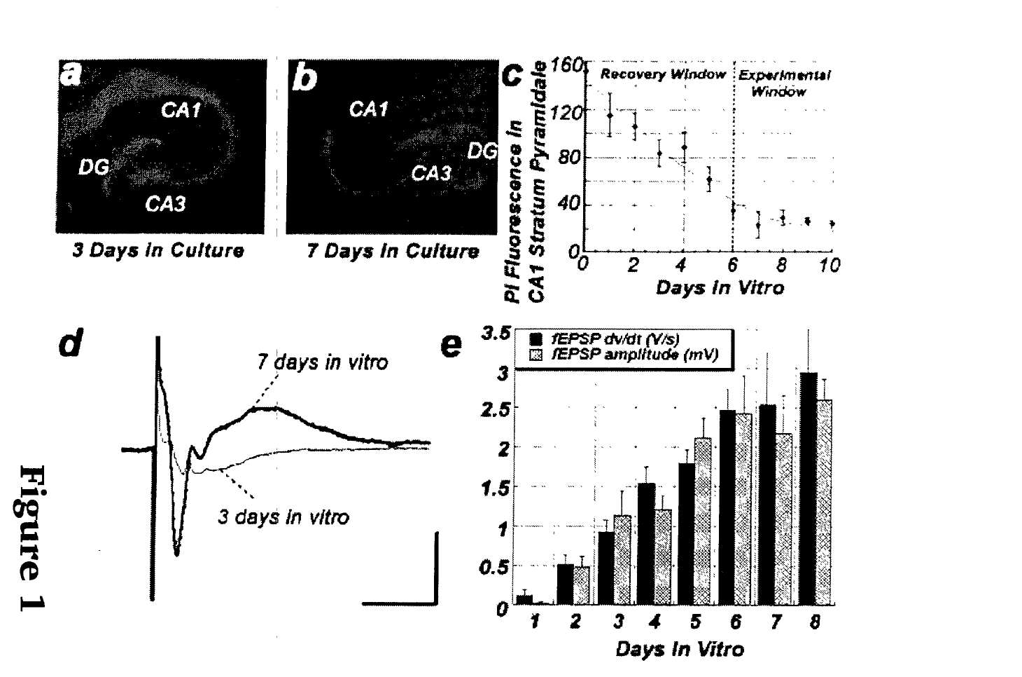

Initial studies characterized the time course of recovery from damage due to slice culture preparation using both anatomical and functional criteria. Measurements of the extent of neuronal death in hippocampal principal layers using PI staining each day for the first 10 days in culture (DIC), illustrated in representative slice cultures in FIG. 1a (3 DIC) and 1b (7 DIC) and summarized in FIG 1c, indicated that the high cell death initially present in slice cultures progressively decreased during the first 5 DIC (“the recovery window”), so that by 6-10 DIC, cell death was at a low steady-state level. Recordings of Schaffer collateral-evoked field excitatory postsynaptic potentials during each of the first 10 DIC revealed a similar time course of functional stabilization, as indicated in FIG. 1d (representative excitatory postsynaptic potentials recorded on days 3 and 7 in culture) and FIG. 1e (summary data on excitatory postsynaptic potentials rise time and amplitude from 6 different slice cultures examined at each day of culture). Based on both PI staining and excitatory postsynaptic potential data, we limited our studies to slice cultures at 6-10 DIC (“experimental window”) (FIG. 1c).

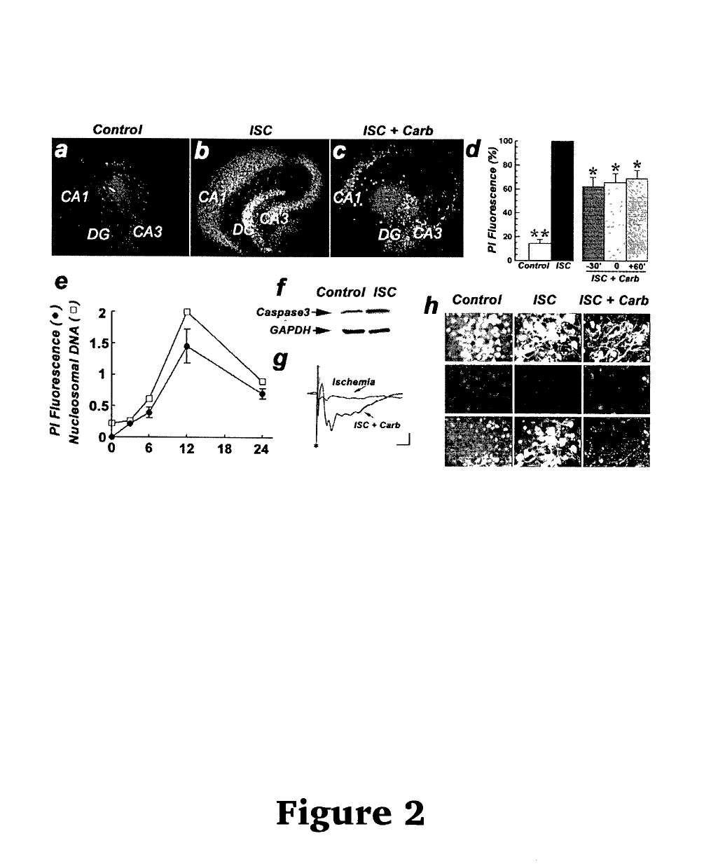

Sustained hypoxia produced severe and irreversible functional deficits at Schaffer collateral-CA1 synapses in vitro and lead to widespread delayed neuronal death in all principal hippocampal layers. To produce consistent hypoxic-ischemic injury to CA1 pyramidal neurons, slice cultures were subjected to 45 min of 95% N₂/5% CO₂+glucose deprivation, which led to marked PI uptake measured 24 hr later (FIG. 2b).

Consistent with the hypothesis that gap junctions contribute to delayed neuronal death, carbenoxolone (75 μM) added to the culture medium 30 min prior to, during and 60 min after application of a 45 min HI insult markedly reduced the delayed death of CA1 pyramidal neurons assessed by PI staining intensity 24 hr following hypoxic-ischemia (FIG. 2c and 2d). Treatment of control organotypic slice cultures with carbenoxolone alone did not detectably alter the viability or morphology of slices 24 hr later (data not shown). The time course of PI staining in the first 24 hr post-HI in stratum pyramidale of field CA1 (FIG. 2e) is suggestive of apoptosis, a conclusion reinforced by assaying in parallel the generation of nucleosomal DNA fragmentation in these slices (FIG. 2e) and activated caspase-3 (FIG. 2f).

Consistent with its actions to reduce death of CA1 pyramidal neurons, carbenoxolone was very effective in protecting against the loss of synaptic transmission in CA1 stratum radiatum normally occurring 24 hr post-HI. FIG. 2g illustrates these experiments on two typical slices, in which HI caused almost complete loss of synaptic transmission, as measured by initial excitatory postsynaptic potential slope, which did not recover in untreated slice cultures 24 hrs following ischemia. A second group of hippocampal slices treated with 75 μM carbenoxolone demonstrated substantial neuroprotection as illustrated by recovery of evoked excitatory postsynaptic potentials (ISC+Carb) after identical 45 min HI episodes.

To directly determine whether ischemia and/or carbenoxolone altered functional intercellular gap junctional coupling in hippocampal slice cultures, we used the fluorescence recovery after photobleaching (FRAP) technique. FIG. 2h illustrates areas within stratum pyramidale of field CA1 subjected to FRAP under control conditions and at 24 hr following ischemia with and without carbenoxolone pre-treatment. The top row (FIG. 2h) illustrates carboxyfluorescein-loaded cells; the middle row (FIG. 2h) at 2 min and the bottom row (FIG. 2h) at 9 min after photobleaching. Quantitation of FRAP experiments indicated that recovery was significantly more rapid following HI than in normoxic slices and that carbenoxolone applied to ischemic cultures 45 min prior to FRAP assays decreased the rate of recovery (FIG. 2h, (ISC+Carb)). These data provide evidence that functional gap junctional coupling is substantially increased at 24 hr post-HI in hippocampal slice cultures and confirm that 75 mM carbenoxolone is effective at reducing gap junction coupling in slice cultures.

### EXAMPLE 2

### Neuroprotection Afforded by Carbenoxolone In Vivo.

In this Example, we show that the efficacy of neuroprotection afforded by carbenoxolone treatment in vitro can be applied to an in vivo model which in turn suggests that pharmacological intervention to block gap junctional channels, during or even after hypoxic-ischemic insult, might improve recovery and lessen brain damage in vivo, especially at times early in development when expression of neuronal gap junctions is particularly high.

Methods

Sprague Dawley fetuses.

Sprague Dawley fetuses were subjected to transient global anoxia during C-section delivery (Bjelke et al., 1991; Brake et al., 1997). Pregnant rats at term (21 days gestation) were decapitated, the abdomen incised, and the uterus was quickly isolated from its blood supply and surrounding fat tissue and connective tissue. Acute anoxia was induced by immersing the intact uterus containing the fetuses into 37° C. saline for 12.5-15 min. Surgical survival was 92±5% following 12.5 min of birth anoxia. Of the 38 surgical C-sections performed, 40% of the surrogate dams accepted the litter mates. To minimize variations between different groups of untreated and carbenoxolone treated HI pups, pups were compared from the same litter; controls were quickly delivered by C-section without additional hypoxicischemia. Carbenoxolone (75 mg/kg; ˜150 μM), was administered i.p twice within 24 hrs of intrauterine hypoxic-ischemia, once (75 mg/kg) immediately after recovery of respiration, the second (30 mg/kg) 12 hrs later, at concentrations known to be effective in blocking gap junction channels in vivo. Blood gases and pH Samples were analyzed for blood gases and pH on a blood gas monitor (RapidLab 855, Bayer,UK). Carbenoxolone has been shown to cross both the normoxic foeto-placental and adult blood-brain barriers following intraperitoneal (i.p.) and subcutaneous injections (Welberg et al., 2000; Jellinck et al., 1993).

Animals were deeply anesthetized with ketalar and perfused via a transcardiac site after which brains were removed, rinsed with saline and fixed in formaldehyde buffered to pH 7.35. Brains were cut into sagittal sections, sliced in 2 mm cross-sections (Braintree Scientific Inc, Mass.), dehydrated with a tissue processor (TP1050, Leica Instruments, Germany), embedded in paraffin, sectioned with a Leica freezing microtome, and stained with haematoxylon-eosin.

Groups were compared by ANOVA or Student's t-test, as appropriate, with group size pre-selected based on P<0.05 significance criterion.

Results and Conclusions

Blood gases were measured immediately after pups, subjected to hypoxia-ischemia, were delivered by C-section and showed a drop from pH=7.3±0.1 (control with C-section) to pH=6.8±0.2 (hypoxic-ischemia group). Furthermore, pO₂ dropped from 76±3.5 mm Hg (control with C-section) to 45±5 mm Hg (hypoxic-ischemia group)(n=8).

Across all litters, intra-uterine hypoxic-ischemia resulted in 36.5%, 43% and 49.5% death in untreated pups after 4, 24 and 48 hrs of reoxygenation/ recirculation, respectively. In contrast, mortality was 23.5%, 27% and 30% in carbenoxolone-treated (75 mg/Kg) ischemic litters at these times following perinatal HI (P=0.02, Fisher exact test, 48 hr survival HI vs. carbenoxolone-treated HI pups). 76.5±9%, 73±9.5% and 70±12.5% of the carbenoxolone-treated (75 mg/Kg) ischemic rats survived reperfusion at these times. By 21 days post-HI, the survival rate was 45.5±15% for untreated animals compared to 70±12% for carbenoxolone-treated HI rat pups (P=0.14, Fisher-exact test).

In accord with previous studies (Rice et al., 1981; Towfighi et al., 1995; Trescher et al., 1990) pathology resulting from intra-uterine hypoxiaischemia varied between litters, ranging from slowed growth to gross encephalopathy, including developmental abnormalities in the hippocampus, neocortex and cerebellum.

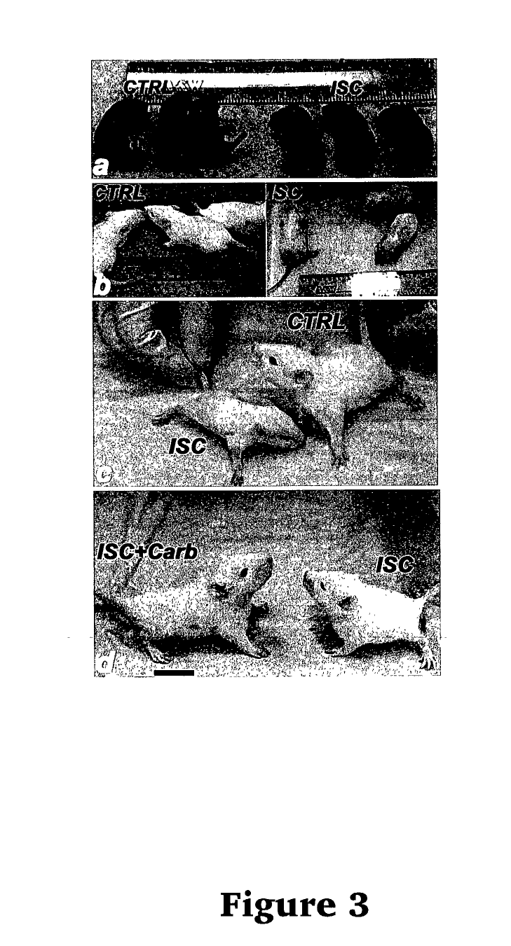

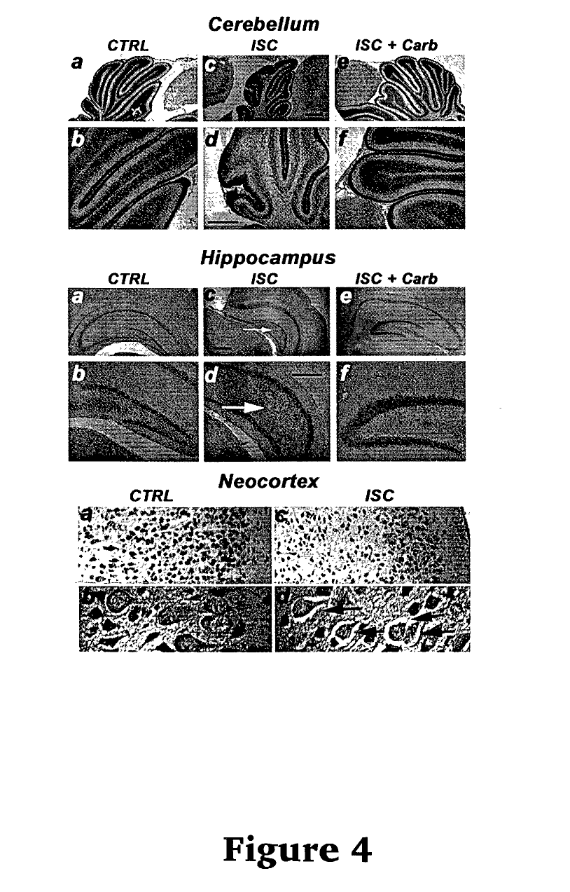

On postnatal day 21, 16% of HI litters weighed about 40% and 20% less than normoxic and hypoxic-ischemic carbenoxolone-treated litter mates, respectively (FIG. 3b-d; P<0.005; Control=38.5±1 g; hypoxicischemic group=23±4 g; carbenoxolone-treated HI rats=29.2±3 g). Histopathological analysis of haematoxylon-eosin stained sections from five ischemic P9-P21 brains from different rats subjected to perinatal hypoxicischemia compared to controls and carbenoxolone-treated litter mates, revealed that 40% showed hypercellularity in the hilar region of the hippocampal dentate gyrus, a site of late neurogenesis (FIG. 4, Hippocampus, Panels c and d, ISC; white arrow). 60% expressed hypercellularity and thickness abnormalities in molecular layers of the cerebellum (FIG. 4, Cerebellum). 40% exhibited pyknotic neocortical neurons with damaged membranes suggestive of apoptotic death, and disorganization of cortical laminae (FIG. 4, Neocortex). 20% expressed abnormal cerebellar and hippocampal growth and 60% showed ventricular dilation (not shown). The functional consequences of this damage and the extent to which ischemic pups ever developmentally “catch up” to their peer groups remains unknown.

To test the hypothesis that gap junctional coupling may be a contributor to these long-term consequences the effect of carbenoxolone treatment was evaluated. Carbenoxolone dramatically reduced the extent of histopathological damage in the hippocampus (FIG. 4, Cerebellum, Panels e and f, “ISC+Carb”) and neocortex (FIG. 4, Neocortex, Panels c and d “ISC”) (n=5), as well as preventing cerebellar damage (FIG. 4, Cerebellum, Panels e and f “ISC+CARB”). Carbenoxolone does not appear to interfere with synaptic function or intrinsic neuron membrane properties i.e., action potentials, spontaneous activity and resting conductance (Travagli et al., 1995; Schmitz et al., 2001).

In addition, our data demonstrates that carbenoxolone can be administered i.p. to newborn rats at least 15 min after HI, and still cross the blood-brain barrier in sufficient time and concentration to markedly reduce the long-term damage induced. Moreover, the survival of these animals also demonstrates that a concentration of carbenoxolone can be used that avoids the well-known cardiac toxicity of gap junction blockers (Rozental et al., 2001a).

### EXAMPLE 3

### Gap Junction Blockade and Caspase-3

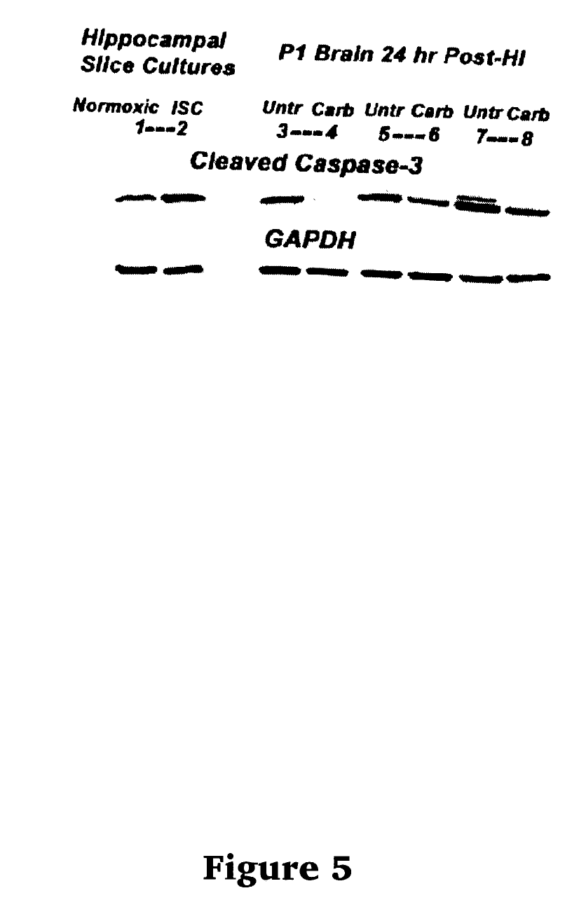

In this Example, we assessed quantitatively the effects of hypoxic-ischemia and gap junction blockade after hypoxic-ischemia on activation of the apoptosis-triggering enzyme caspase-3, by performing western blot assays on hippocampal slice cultures 24 hr. following hypoxic-ischemia (FIG. 5).

Western Blot Analysis.

Protein was extracted from whole-brain 24 hr following HI and concentration determined with a BCA Protein Assay Kit (Pierce, Rockford, Ill.). Twenty-five μg protein/sample was applied per lane and separated by electrophoresis in 10% or 10-20% SDS-polyacrylamide gels (BioRad, Hercules, Calif.) and transferred to nitrocellulose membranes (Schleicher and Schuell, Keene, N.H.).

Membranes were blotted at RT with cleaved caspase-3 (Asp 175) antibody (1:700) overnight at 4° C., and then incubated in horseradish peroxidase-conjugated anti-rabbit IgG 1:1500. Signals were revealed with chemiluminescence reagents (ECL; Amersham Pharmacia Biotech, Piscataway, N.J.) and visualized by autoradiography. As an internal control, glyceraldehyde-3-phosphate dehydrogenase (GAPDH) was blotted with horseradish peroxidase-conjugated goat antimouse IgG at a concentration of 1:4000. Densitometriy of chemiluminescent bands was quantified using Scion Image densitometer software (NIH), and band densities normalized to GAPDH density in the same gel.

In hippocampal slice cultures following hypoxic-ischemia, activated caspase-3 protein abundance increased to 192±9% ( n=24 slices from 3 litter mates; P<0.005; control vs. HI) (FIG. 5). Consistent with these data, activated caspase-3 protein abundance was also significantly increased in whole brains from HI-treated newborn rats, an effect that was largely prevented by carbenoxolone treatment immediately after HI. In all three experiments, carbenoxolone administered i.p. (75 mg/kg) immediately after perinatal hypoxia-ischemia reduced to 43±15% the activation of caspase-3 24 hr post-HI (P=0.05; band densities: HI=683±97, HI+Carb=302±115). This example confirmed the finding that carbenoxolone administration (gap junction blockade) inhibits activation of caspase-3 by intrauterine hypoxic-ischemia.

The normal occurrence of programmed cell death (Sidhu et al., 1997) and caspase-3 expression (Siman et al., 1999) in the developing brain in the absence of HI may modulate the death of immature neurons after perinatal HI. In the present study, HI activation of caspase-3 was demonstrated by the appearance of the active p17 subunit (17 kD) on both organotypic slice cultures and P1 ischemic brains. Although it was not shown directly that this caspase-3 activity caused the eventual neuronal death, the pronounced expression of p17 subunits in P1 brains preceded delayed neuronal death 24 hr later, and the widespread neuropathology present at P9 and P21. Moreover, carbenoxolone treatment dramatically reduced caspase-3 activation, delayed neuronal death and eventual long-term brain damage in a correlated fashion, consistent with a causal connection between cell coupling, activation of apoptotic pathways and eventual neuronal death.

### EXAMPLE 4

### Delayed Neuronal Death Due to Apoptosis

In this Example, we assessed the amount of delayed neuronal death due to apoptosis by comparing the time course of PI fluorescence with the appearance of nucleosomal DNA fragments in whole hippocampal slice cultures over the first 24 hr following hypoxic-ischemia thereby evaluating the nature of neuronal death following hypoxic-ischemic injury. Although many studies suggest that the majority of neuronal loss 24 hr post-HI is apoptosis-like, necrosis could also contribute to the delayed neuronal death (Pitts, 1994). Both DNA fragmentation and caspase activation are hallmarks of apoptosis. The appearance of nucleosomal DNA fragments correlates with the complete collapse of chromatin structure and the formation of apoptotic bodies.

ELISA Detection of Nucleosomes.

Apoptotic cell death was assessed using a commercially available ELISA for the detection of nucleosomes (Boehringer Mannheim). Filters containing slice cultures were incubated in 300 μl of lysis buffer for 30 min at room temperature, centrifuged, and supernatant collected. Aliquots were sampled at each time point after HI and protein content normalized using the Bradford assay (Bio-Rad). Samples were diluted to equal protein concentration, and 20 μl of normalized sample incubated with immunoreagent (a mix of peroxidase-conjugated anti-DNA antibody and biotin-conjugated anti-histone antibody) in a streptavidin-coated microtiter plate. After shaking for 2 hr at room temperature, the plate was washed and reacted with the chromogenic substrate. A Thermomax 96-well plate reader (Molecular Devices) was used to read absorbance at 490 nm, which was subtracted from the absorbance at 405 nm, and nucleosomal signal expressed as normalized relative units.

Nucleosomal DNA fragmentation began to rise by 3 hr post-HI, with a marked 20-fold peak increase by 12 hr (FIG. 2f). PI fluorescence showed a similar time course of hippocampal cell death in CA1 stratum pyramidale, with peak dead cell fluorescence 12 hr post-HI (FIG. 2e). The decrease in nucleosome signals after 12 hr appears to be due to a rate of nucleosome degradation that overtakes the rate of nucleosome production. The strong temporal correlation between these two measures indicates that an apoptosis-like process is rapidly induced in hippocampus after hypoxic-ischemia and is probably the major contributor to neuronal loss.

### EXAMPLE 5

### Effect of Gap-Junction Blockers on Seizures

In this Example, we establish that carbenoxolone stops the behavioral and electrographic seizures induced by intra-uterine hypoxic-ischemia. In addition, the histopathological consequences of hypoxic-ischemia induced seizures were also examined. The intrauterine hypoxic-ischemia model is particularly well suited for the controlled application of hypoxic-ischemia insults. An advantage of this model is that the brain insult is homogeneous among different neonatal rats from the same litter.

Intra-uterine Hypoxic-ischemia.

Rat pups were delivered by C-section. One group was subjected to intra-uterine hypoxic-ischemia, the control group was also delivered by caesarian-section but not subjected to a period of hypoxic-ischemia. Both groups were observed for the occurrence of seizures. EEG was performed on one pup before and 40 min after administration of carbenoxolone.

Histology Examination.

Surviving animals in all groups were examined neuropathologically at 21 days following delivery (P21).

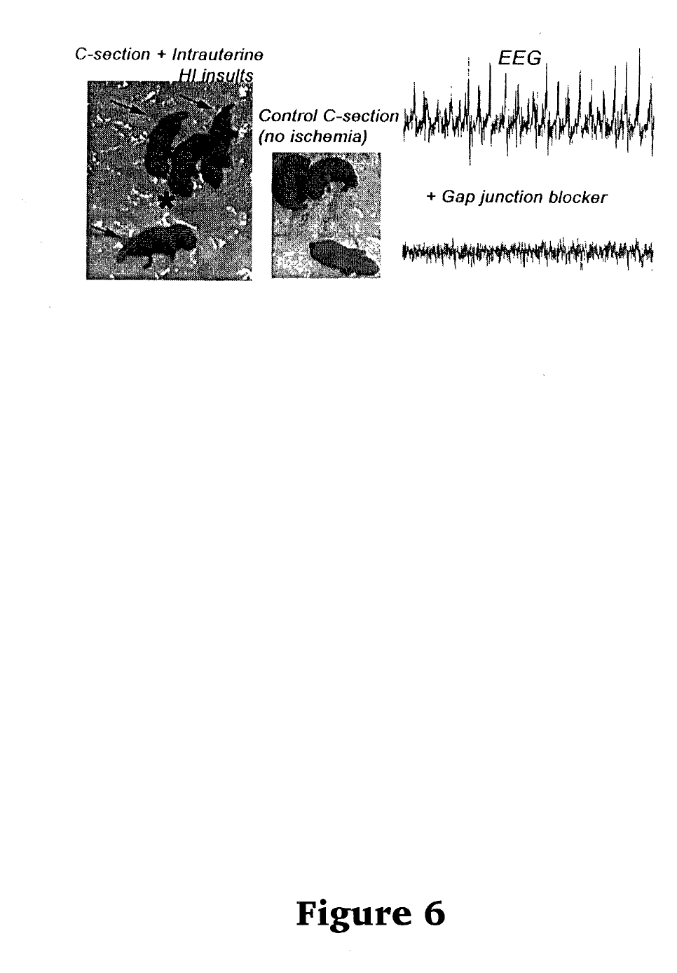

Under our experimental conditions, approximately 30% of the neonatal rats (n=100) expressed behavioral seizures following intrauterine HI insults. Behavioral seizures were quite stereotyped and diagnosed as a tonic posturing with both the forelimbs and hind limbs stiffly extended alternating with periods of “swimming-like” activity (FIG. 6A, black arrows). Typically, behavioral seizures were evident by 30 to 60 min following delivery of ischemic rats and lasted from 1 to 4 hrs. EEG studies obtained to assess the morphology and frequency of the corresponding electrographic seizures revealed marked changes from baseline due to an increase in frequency and rhythmicity of the discharges. Intrauterine HI insults evoke behavioral seizures in neonatal rats 30-60 min following delivery. Approximately 30% of ischemic pups undergo tonic extensions (FIG. 6a, black arrows) and/or “swimming-like” activity (FIG. 6a). Behavioral seizures are not observed in controls (C-section but no period of added ischemia) (FIG. 6b). In addition, differences in postural behavior between ischemic versus control groups were observed (not shown).

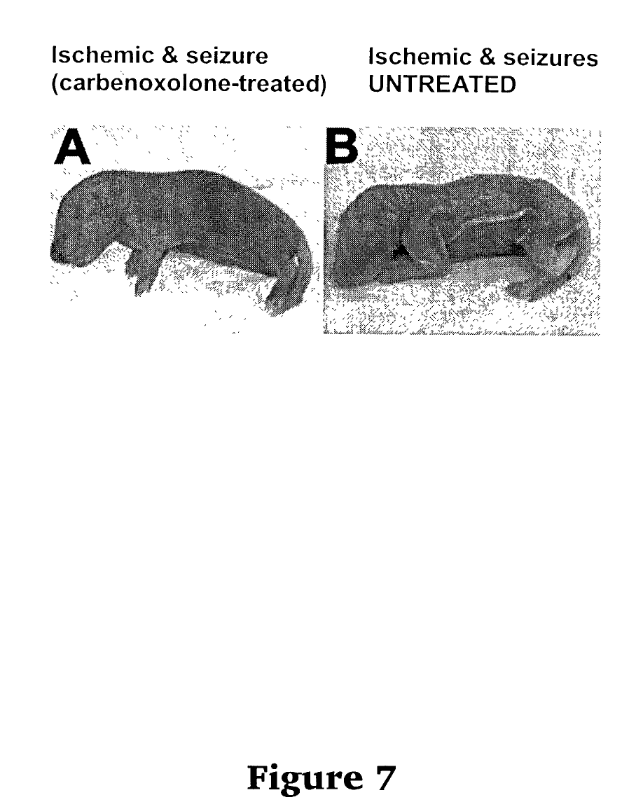

The gap junction blocker, carbenoxolone, administered i.p. at the onset of motor seizures abolishes behavioral seizures following HI insults. In the untreated rat, repetitive and prolonged behavioral seizures were present for at least 4 hr and, in contrast to the treated pup, the untreated rat maintains a different posture and is dehydrated (FIG. 7B). Samples of EEGs obtained during behavioral seizures are illustrated in FIG. 6. The effects on electrographic seizures is illustrated by EEG recordings on one single pup before (FIG. 6, EEG) and 40 min after administration of carbenoxolone i.p. (FIG. 6, + Gap junction blocker). In order to complete the electroencephalogram, rats were immobilized to prevent movement artifacts.

The mortality rate in rats with repetitive behavioral seizures within the first 24 hrs post-delivery was 50% (n=15). Carbenoxolone stopped the behavioral and electrographic seizures. One of 12 ischemic pups treated with carbenoxolone (75 μM) died.

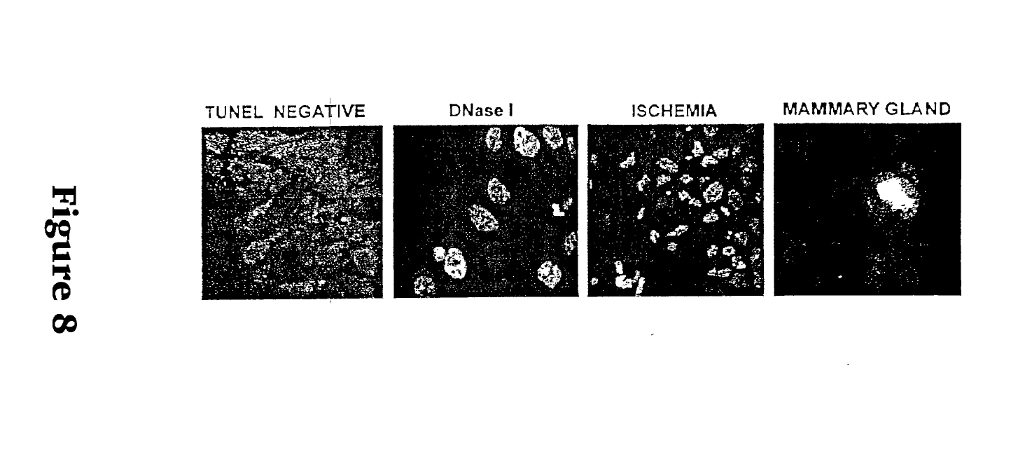

The neuropathologic effects of perinatal ischemia and seizures on hippocampus at 21 days was examined (FIG. 8). Considerable hippocampal damage was observed in P21 rats following H/I insults combined with behavioral seizures. Neuropathological outcome reveal both cortical (FIG. 4, NEOCORTEX) and hippocampal damage. Intra-uterine ischemia was maintained for 15 minutes. Blood gases measured immediately after delivery by C-section revealed a drop from pH 7.3±0.1 (control; C-section with no period added of hypoxia/ischemia) to pH 6.8±0.2 (C-section following a period of hypoxic-ischemia). TUNEL staining (FIG. 8), an assay to assess apoptotic cell death, shows pycnotic nuclei in P21 hippocampus following global brain ischemia (FIG. 8, ISCHEMIA). Ischemic hippocampus with the omission of TdT from the TUNEL mix is used as an “internal negative” control (FIG. 8, TUNEL NEGATIVE). Control hippocampus (non-ischemic) treated with DNase is illustrated as an “internal positive” control (FIG. 8, DNase I). Control involuting mammary gland of mothers three days following removal of the pups is used as an additional “external positive” control and shows the round chromatin clumps typical of apoptotic cells (FIG. 8, MAMMARY GLAND).

Consistent with our hypothesis that coupling plays a role in delayed neurodegeneration by mediating electrical synchrony between neurons, underlying seizures, and metabolic cooperation between apposed cells, carbenoxolone (75 μM) confers marked neuroprotection when applied in vitro and abolishes behavioral and electrographic seizures when applied in vivo (FIG. 6, EEG+Gap junction blocker).

In both hippocampal organotypic slice cultures and in intrauterine hypoxic-ischemia in vivo, it was found that carbenoxolone treatment was neuroprotective, even when administered following the ischemic events. This is consistent with the fact that intercellular communication through gap junction channels during brain hypoxic-ischemia provides a pathway for propagation of neuronal damage and death (Cotrina et al., 1998; Lin et al., 1998). The FRAP data showing an increase in coupling in organotypic slice cultures 24 hr post-ischemia (FIG. 2h) is consistent with a recent report using an in vitro trauma model and indicates that hypoxic-ischemia leads to a situation even more favorable for bystander cell killing (Frantseva, et al., 2002a).

In view of the above, it will be seen that the several advantages of the invention are achieved and other. advantages attained. As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method for preventing a perinatal hypoxic-ischemic cerebral injury in an infant, the method comprising administering to a woman pregnant with the infant, or to the infant following labor and delivery an amount of gap junction blocker effective to prevent hypoxic-ischemic cerebral injury in the infant.

2. The method of claim 1, wherein the gap junction blocker is administered to the woman.

3. The method of claim 1, wherein the gap junction blocker is administered to the infant.

4. The method of claim 1, wherein the gap junction blocker is administered to the woman during labor.

5. The method of claim 1, wherein the gap junction blocker is administered to the woman before labor.

6. The method of claim 1, wherein the woman has a high risk pregnancy.

7. The method of claim 4, wherein the woman undergoes term labor.

8. The method of claim 4, wherein the woman undergoes preterm labor.

9. The method of claim 8, wherein the infant is preterm.

10. The method of claim 8, wherein the infant is term.

11. The method of claim 1, wherein the delivery is vaginal.

12. The method of claim 1, wherein the delivery is by caesarian section.

13. The method of claim 1, wherein the hypoxic-ischemic cerebral injury is suspected or diagnosed.

14. The method of claim 1, wherein the gap junction blocker is selected from the group consisting of fluothane, ethrane, heptanol and a glycyrrhetinic acid derivative.

15. The method of claim 1, wherein the gap junction blocker is a glycyrrhetinic acid derivative.

16. The method of claim 15, wherein the gap junction blocker is carbenoxolone.

17. The method of claim 1, wherein the hypoxic-ischemic cerebral injury is selected from the group consisting of stroke, focal ischemic injury and global ischemic injury.

18. The method of claim 17, further comprising a second hypoxic-ischemic cerebral injury.

19. The method of claim 1, wherein the hypoxic-ischemic cerebral injury is diagnosed by a method selected from the group consisting of a clinical exam, MRI, CT scan, PET scan, X-ray, arteriography, electroencephalograph, blood gas examination, blood electrolyte examination and cerebro-spinal fluid examination.

20. The method of claim 1, wherein the infant has epilepsy, seizures or abnormal neurologic development.

21. The method of claim 1, wherein the hypoxic-ischemic cerebral injury is a result of a condition selected from the group consisting of anterior circulation stroke, cerebral venous thrombosis, West syndrome, lysosomal storage disease, Fabry disease, homocystinuria, MELAS, neuronal ceroid lipofuscinosis, peroxisomal disorders, hyperammonemia, hypocalcemia, methylmalonic acidemia, ornithine transcarbamylase deficiency, propionic acidemia, epileptogenic encephalopathy, intractable seizures, perinatal asphyxia, encephalitis, meningitis, birth trauma, tuberous sclerosis, congenital cerebral malformations, intracranial tumors, intracranial hemorrhage, toxoplasmosis, rubella, syphilis, cytomegallovirus, herpes, systemic metabolic disease, Lennox-Gastaut syndrome, epileptogenic encephalopathy, intractable seizures, perinatal asphyxia, encephalitis, meningitis, birth trauma, tuberous sclerosis, congenital cerebral malformations, intracranial tumors, intracranial hemorrhage, toxoplasmosis, rubella, syphilis, cytomegallovirus, herpes, systemic metabolic disease and cephalo-pelvic disproportion.

22. The method of claim 1, wherein the gap junction blocker is administered to the woman by a method selected from the group consisting of oral, intravenous, intra-arterial, rectal, nasal, dermal and vaginal.

23. The method of claim 1, wherein the gap junction blocker is administered to the infant by a method selected from the group consisting of oral, intravenous, intra-arterial, rectal, nasal, dermal and into the cerebro-spinal fluid.

24. A method for preventing seizures induced by an hypoxic-ischemic injury in an infant, the method comprising administering to a woman pregnant with the infant, or to the infant following labor and delivery an amount of gap junction blocker effective to prevent seizures following hypoxic-ischemic injury in an infant.

25. The method of claim 24, wherein the seizures are postnatal seizures.

26. The method of claim 24, wherein the woman undergoes term labor.

27. The method of claim 24, wherein the woman undergoes preterm labor.

28. The method of claim 24, wherein the seizures are clinical.

29. The method of claim 24, wherein the seizures are electrographic.

30. The method of claim 24, wherein the seizures are selected from a group consisting of focal, partial, generalized, tonic, atonic, myoclonic, astatic, bicycling, minor motor and apnoeic seizures.

31. The method of claim 24, wherein the gap junction blocker is selected from a group consisting of fluothane, ethrane, heptanol and glycyrrhetinic acid derivatives.

32. The method of claim 24, wherein the gap junction blocker is a glycyrrhetinic acid derivative.

33. The method of claim 32, wherein the gap junction blocker is carbenoxolone.

34. The method of claim 24, wherein the gap junction blocker is administered to the infant by a method selected from a group consisting of oral, intravenous, intra-arterial, rectal, nasal, dermal and vaginal.

35. The method of claim 24, wherein the gap junction blocker is administered to the woman during labor.

36. The method of claim 35, wherein the gap junction blocker is administered to the woman by a method selected from a group consisting of oral, intravenous, intra-arterial, rectal, nasal, dermal and vaginal.

37. The method of claim 24, wherein the gap junction blocker is administered to the woman before labor.

38. The method of claim 37, wherein the gap junction blocker is administered to the woman by a method selected from the group consisting of oral, intravenous, intra-arterial, rectal, nasal, dermal and vaginal.

39. A method for treating seizures induced by hypoxic-ischemic cerebral injury in a infant, the method comprising administering to a woman pregnant with the infant or to the infant following labor and delivery an amount of gap junction blocker effective to treat seizures following hypoxic-ischemic injury in a preterm or term infant.

40. The method of claim 39, wherein the seizures are postnatal seizures.

41. The method of claim 39, wherein the labor is term.

42. The method of claim 39, wherein the labor is preterm.

43. The method of claim 39, wherein the seizures are clinical.

44. The method of claim 39, wherein the seizures are electrographic.

45. The method of claim 43, wherein the seizures are selected from a group consisting of focal, partial, generalized, tonic, atonic, myoclonic, astatic, bicycling, minor motor, apnoeic seizures and status epilepticus.

46. The method of claim 39, wherein the gap junction blocker is selected from a group consisting of fluothane, ethrane, heptanol and glycyrrhetinic acid derivatives.

47. The method of claim 39, wherein the gap junction blocker is a glycyrrhetinic acid derivative.

48. The method of claim 39, wherein the gap junction blocker is carbenoxolone.

49. The method of claim 39, wherein the gap junction blocker is administered to the infant.

50. The method of claim 49, wherein the gap junction blocker is administered to the infant by a method selected from a group consisting of oral, intravenous, intra-arterial, rectal, nasal, dermal and into cerebro-spinal fluid.

51. The method of claim 39, wherein the gap junction blocker is administered to the woman during labor.

52. The method of claim 51, wherein the gap junction blocker is administered to the woman by a method selected from a group consisting of oral, intravenous, intra-arterial, rectal, nasal, dermal and vaginal.

53. The method of claim 39, wherein the gap junction blocker is administered to the woman before labor.

54. The method of claim 53, wherein the gap junction blocker is administered to the woman by a method selected from a group consisting of oral, intravenous, intra-arterial, rectal, nasal, dermal and vaginal.

56. A kit comprising a gap junction blocker and instructions for preventing perinatal hypoxic-ischemic cerebral injury in an infant by administering to a woman pregnant with the infant during labor or to the infant following delivery an amount of gap junction blocker effective to prevent hypoxic-ischemic cerebral injury in the infant.

57. The kit of claim 56, further comprising an apparatus for administration.

58. The kit of claim 56, wherein the gap junction blocker is carbenoxolone.

59. A method of preventing hypoxic-ischemic injury in a neuron in an infant comprising administering to a woman pregnant with the infant during labor or to the infant following delivery an amount of gap junction blocker effective to prevent hypoxic-ischemic injury to the neuron.

60. The method of claim 59, wherein the gap junction blocker is carbenoxolone.

61. A method for conferring neuro-protection to an individual following head trauma, the method comprising administering to the individual an amount of gap junction blocker effective to confer neuro-protection following head-trauma in the individual.

62. The method of claim 61, wherein the gap junction blocker is selected from a group consisting of fluothane, ethrane, heptanol and glycyrrhetinic acid derivatives.

63. The method of claim 61, wherein the gap junction blocker is a glycyrrhetinic acid derivative.

64. The method of claim 61, wherein the gap junction blocker is carbenoxolone.

65. The method of claim 61, wherein the gap junction blocker is administered by a method selected from oral, nasal, intra-arterial, intravenous and into the cerebro-spinal-fluid.
